(19) United States
(12) Patent Application Publication
(10) Pub. No.: US 2015/0017670 A1
(43) Pub. Date: Jan. 15, 2015

(54) Methods For Determining The Degradation Of A Biomass Material

(71) Applicant: Novozymes, Inc., Davis, CA (US)

(72) Inventors: Jason Quinlan, Albany, CA (US); Brett McBrayer, Sacramento, CA (US); Elena Vlasenko, Davis, CA (US); David Osborn, Sacramento, CA (US)

(21) Appl. No.: 14/367,194

(22) PCT Filed: Dec. 20, 2012
(86) PCT No.: PCT/US2012/071006

§ 371 Date: Jun. 19, 2014

Related U.S. Application Data
(60) Provisional application No. 61/578,809, filed on Dec. 21, 2011.

(51) Int. Cl. C12Q 1/34

(52) U.S. Cl. CPC C12Q 1/34; G01N 2333/942
USPC 435/18; 435/288.4

(57) ABSTRACT

The present invention relates to methods for analyzing degradation of a biomass material at a high dry weight percentage.

### STATEMENT AS TO RIGHTS TO INVENTIONS MADE UNDER FEDERALLY SPONSORED RESEARCH AND DEVELOPMENT

This invention was made with Government support under Cooperative Agreement DE-FC36-08GO18080 awarded by the Department of Energy. The government has certain rights in this invention.

### BACKGROUND OF THE INVENTION

1. Field of the Invention

The present invention relates to methods for determining the degradation of a biomass material at a high dry weight percentage.

2. Description of the Related Art

Biomass is used as a renewable energy source for the production of biofuel. Industrial biomass can be grown from numerous types of plants, including miscanthus, switchgrass, hemp, corn, poplar, willow, sorghum, sugarcane, and a variety of tree species, ranging from eucalyptus to oil palm.

The conversion of lignocellulosic feedstocks into ethanol has the advantages of the ready availability of large amounts of feedstock, the desirability of avoiding burning or land filling the materials, and the cleanliness of the ethanol fuel. Wood, agricultural residues, herbaceous crops, and municipal solid wastes have been considered as feedstocks for ethanol production. These materials primarily consist of cellulose, hemicellulose, and lignin. Once the lignocellulose is converted to fermentable sugars, e.g., glucose, the fermentable sugars are easily fermented by yeast into ethanol.

Current methods of analyzing, evaluating, or screening the hydrolysis or degradation activity of an enzyme composition toward a biomass material are problematic and not well designed for high-throughput analysis at high dry weight percentages. For example, one method of analyzing the hydrolysis of pretreated corn stover (hereinafter referred to as “PCS”) at high dry weight percentages requires weighing out a suitable mass of PCS, water, buffer and cellulolytic enzyme(s) into a flask. The cellulose in the PCS is enzymatically hydrolyzed to glucose, cellobiose, and higher oligosaccharides. HPLC is used to measure the glucose and cellobiose. One skilled in the art, knowing the cellulose content of the substrate, can then use this information to calculate the percent conversion of cellulose into sugars. Accordingly, the cellulolytic activity of the enzymes in the hydrolysis can be measured. However, the weighing step is time-consuming and laborious, and the physicochemical properties of high solids PCS slurries prevent the automation of this step, precluding the ability to assay in high or even medium throughput. Consequently, PCS must be milled and diluted to permit pipetting, or must be assayed in a low throughput manner. Assays using high solids PCS or other lignocellulosic biomasses are not suitable for high-throughput analysis, and/or a quick analysis of multiple enzymes of interest in a single assay.

Current assays lead to higher research costs, tedious assay formatting, and time-consuming enzyme activity evaluation. Accordingly, there is a continuing need for less expensive and time-consuming assays and analysis methods having improved accuracy, especially where high-throughput analysis is desirable.

It is widely known that industrial cellulosic ethanol production is likely to take place at high solids, thereby generating sufficient ethanol for economic feasibility. It would be advantageous in the art to develop an assay that will most closely approximate these conditions to assess enzyme compositions under conditions of high solids (e.g., high lignocellulose content) at higher throughput than is currently available. It is further widely known that grinding or milling lignocellulosic biomass is a costly expense, prohibitive to commercial ethanol production, and therefore unlikely to be performed. It would also be advantageous in the art to develop an assay that does not require substrate milling and can therefore reflect the hydrolysis of pretreated or unpretreated lignocellulosic biomass in unmodified form.

The present invention provides methods for analyzing degradation of a biomass material at a high dry weight percentage.

### SUMMARY OF THE INVENTION

The present invention relates to methods for analyzing degradation of a biomass material, comprising:

(a) spreading a slurry of the biomass material over a multi-well fill plate to fill each well thereof, wherein each well of the multi-well fill plate comprises an aperture corresponding to a paired opening in the same well;

(b) removing excess biomass material from the surfaces of the multi-well fill plate;

(c) transferring the biomass material from the wells of the multi-well fill plate to corresponding wells of a multi-well reaction plate by a means that displaces the biomass material from the opening of each well of the multi-well fill plate into the corresponding opening of each well of the multi-well reaction plate, wherein the multi-well reaction plate comprises wells that are larger in volume and larger or equivalent in diameter or width compared to the multi-well fill plate;

(d) adding an enzyme composition to each well of the multi-well reaction plate;

(e) incubating the reaction plate for a period of time at a pH and a temperature for the enzyme composition to degrade the biomass material; and

(f) detecting a signal resulting from the degradation of the biomass material, wherein an increase or a decrease in intensity of the signal indicates the amount of biomass material degraded by the enzyme composition.

The present invention also relates to a multi-well fill plate for transferring a slurry of a biomass material to a multi-well reaction plate, wherein each well of the multi-well fill plate comprises an aperture corresponding to a paired opening in the same well.

### BRIEF DESCRIPTION OF THE FIGURES

FIG. 1 shows a schematic diagram of a representative multi-well fill plate.

FIGS. 2A and 2B show hydrolysis of unmilled NREL pretreated corn stover by various cellulase compositions. FIG. 2A: 96-well high solids plate assay; FIG. 2B: 20 g scale assay. White: 50° C.; gray: 60° C.

FIG. 3 shows hydrolysis of unmilled NREL pretreated corn stover by increasing concentrations of cellulase enzyme composition #1 at various total solids. Circles: 10% solids; squares: 12.5% solids; diamonds: 15% solids; triangles: 20% solids.

FIG. 4 shows hydrolysis of 20% total solids unmilled NREL PCS by 2 different cellulase compositions. Open circles: cellulase enzyme composition #2; closed circles: cellulase enzyme composition #3. Data are fit to a modified sigmoidal hydrolysis model.

FIG. 5 shows hydrolysis of 10% total solids milled bleached eucalyptus by 2 different cellulase compositions. Open circles: cellulase enzyme composition #2; closed circles: cellulase enzyme composition #3. Data are fit to a modified sigmoidal hydrolysis model.

FIG. 6 shows hydrolysis of 10% total solids milled unbleached eucalyptus by 2 different cellulase compositions. Open circles: cellulase enzyme composition #2; closed circles: cellulase enzyme composition #3. Data are fit to a modified sigmoidal hydrolysis model.

### DEFINITIONS

Acetylxylan esterase: The term “acetylxylan esterase” means a carboxylesterase (EC 3.1.1.72) that catalyzes the hydrolysis of acetyl groups from polymeric xylan, acetylated xylose, acetylated glucose, alpha-napthyl acetate, and p-nitrophenylacetate. For purposes of the present invention, acetylxylan esterase activity is determined using 0.5 mM p-nitrophenylacetate as substrate in 50 mM sodium acetate pH 5.0 containing 0.01% TWEEN™ 20 (polyoxyethylene sorbitan monolaurate). One unit of acetylxylan esterase is defined as the amount of enzyme capable of releasing 1 μmole of p-nitrophenolate anion per minute at pH 5, 25° C.

Alpha-L-arabinofuranosidase: The term “alpha-L-arabinofuranosidase” means an alpha-L-arabinofuranoside arabinofuranohydrolase (EC 3.2.1.55) that catalyzes the hydrolysis of terminal non-reducing alpha-L-arabinofuranoside residues in alpha-L-arabinosides. The enzyme acts on alpha-L-arabinofuranosides, alpha-L-arabinans containing (1,3)- and/or (1,5)-linkages, arabinoxylans, and arabinogalactans. Alpha-L-arabinofuranosidase is also known as arabinosidase, alpha-arabinosidase, alpha-L-arabinosidase, alpha-arabinofuranosidase, polysaccharide alpha-L-arabinofuranosidase, alpha-L-arabinofuranoside hydrolase, L-arabinosidase, or alpha-L-arabinanase. For purposes of the present invention, alpha-L-arabinofuranosidase activity is determined using 5 mg of medium viscosity wheat arabinoxylan (Megazyme International Ireland, Ltd., Bray, Co. Wicklow, Ireland) per ml of 100 mM sodium acetate pH 5 in a total volume of 200 μl for 30 minutes at 40° C. followed by arabinose analysis by AMINEX® HPX-87H column chromatography (Bio-Rad Laboratories, Inc., Hercules, Calif., USA).

Alpha-glucuronidase: The term “alpha-glucuronidase” means an alpha-D-glucosiduronate glucuronohydrolase (EC 3.2.1.139) that catalyzes the hydrolysis of an alpha-D-glucuronoside to D-glucuronate and an alcohol. For purposes of the present invention, alpha-glucuronidase activity is determined according to de Vries, 1998, J. Bacteriol. 180: 243-249. One unit of alpha-glucuronidase equals the amount of enzyme capable of releasing 1 μmole of glucuronic or 4-O-methylglucuronic acid per minute at pH 5, 40° C.

Beta-glucosidase: The term “beta-glucosidase” means a beta-D-glucoside glucohydrolase (E.C. 3.2.1.21) that catalyzes the hydrolysis of terminal non-reducing beta-D-glucose residues with the release of beta-D-glucose. For purposes of the present invention, beta-glucosidase activity is determined using p-nitrophenyl-beta-D-glucopyranoside as substrate according to the procedure of Venturi et al., 2002, Extracellular beta-D-glucosidase from Chaetomium thermophilum var. coprophilum: production, purification and some biochemical properties, J. Basic Microbiol. 42: 55-66. One unit of beta-glucosidase is defined as 1.0 μmole of p-nitrophenolate anion produced per minute at 25° C., pH 4.8 from 1 mM p-nitrophenyl-beta-D-glucopyranoside as substrate in 50 mM sodium citrate containing 0.01% TWEEN® 20.

Beta-xylosidase: The term “beta-xylosidase” means a beta-D-xyloside xylohydrolase (E.C. 3.2.1.37) that catalyzes the exo-hydrolysis of short beta (1-4)-xylooligosaccharides to remove successive D-xylose residues from non-reducing termini. For purposes of the present invention, one unit of beta-xylosidase is defined as 1.0 μmole of p-nitrophenolate anion produced per minute at 40° C., pH 5 from 1 mM p-nitrophenyl-beta-D-xyloside as substrate in 100 mM sodium citrate containing 0.01% TWEEN® 20.

Biomass material: The term “biomass material” means organic materials from living or recently living organisms, most often plants or plant-derived material. Biomass materials may be used as renewable energy sources such as agricultural and forest residues, dedicated energy crops, municipal solid waste, etc. An example of biomass material is cellulosic material (lignocellulosic biomass). The term lignocellulosic biomass is often used to describe the material that composes the plant cell wall, which includes primarily cellulose, hemicelluloses, and lignin. Additional examples of biomass material are hemicellulosic material, starch material (amyloglucan and amylopectin), and chitinous material.

Cellobiohydrolase: The term “cellobiohydrolase” means a 1,4-beta-D-glucan cellobiohydrolase (E.C. 3.2.1.91 and E.C. 3.2.1.176) that catalyzes the hydrolysis of 1,4-beta-D-glucosidic linkages in cellulose, cellooligosaccharides, or any beta-1,4-linked glucose containing polymer, releasing cellobiose from the reducing end (cellobiohydrolase I) or non-reducing end (cellobiohydrolase II) of the chain (Teeri, 1997, Trends in Biotechnology 15: 160-167; Teeri et al., 1998, Biochem. Soc. Trans. 26: 173-178). Cellobiohydrolase activity is determined according to the procedures described by Lever et al., 1972, Anal. Biochem. 47: 273-279; van Tilbeurgh et al., 1982, FEBS Letters, 149: 152-156; van Tilbeurgh and Claeyssens, 1985, FEBS Letters, 187: 283-288; and Tomme et al., 1988, Eur. J. Biochem. 170: 575-581. In the present invention, the Tomme et al. method can be used to determine cellobiohydrolase activity.

Cellulolytic enzyme or cellulase: The term “cellulolytic enzyme” or “cellulase” means one or more (e.g., several) enzymes that hydrolyze a cellulosic material. Such enzymes include endoglucanase(s), cellobiohydrolase(s), beta-glucosidase(s), or combinations thereof. The two basic approaches for measuring cellulolytic activity include: (1) measuring the total cellulolytic activity, and (2) measuring the individual cellulolytic activities (endoglucanases, cellobiohydrolases, and beta-glucosidases) as reviewed in Zhang et al., Outlook for cellulase improvement: Screening and selection strategies, 2006, Biotechnology Advances 24: 452-481. Total cellulolytic activity is usually measured using insoluble substrates, including Whatman No 1 filter paper, microcrystalline cellulose, bacterial cellulose, algal cellulose, cotton, pretreated lignocellulose, etc. The most common total cellulolytic activity assay is the filter paper assay using Whatman No 1 filter paper as the substrate. The assay was established by the International Union of Pure and Applied Chemistry (IUPAC) (Ghose, 1987, Measurement of cellulase activities, Pure Appl. Chem. 59: 257-68).

For purposes of the present invention, cellulolytic enzyme activity is determined by measuring the increase in hydrolysis of a cellulosic material by cellulolytic enzyme(s) under the following conditions: 1-50 mg of cellulolytic enzyme protein/g of cellulose in PCS (or other pretreated cellulosic material) for 3-7 days at a suitable temperature, e.g., 50° C., 55° C., 60° C., or 65° C., compared to a control hydrolysis without addition of cellulolytic enzyme protein. Typical conditions are 1 ml reactions, washed or unwashed PCS, 5% insoluble solids, 50 mM sodium acetate pH 5, 1 mM MnSO₄, 50° C., 55° C., 60° C., or 65° C., 72 hours, sugar analysis by AMINEX® HPX-87H column (Bio-Rad Laboratories, Inc., Hercules, Calif., USA).

Cellulosic material: The term “cellulosic material” means any material containing cellulose. The predominant polysaccharide in the primary cell wall of biomass is cellulose, the second most abundant is hemicellulose, and the third is pectin. The secondary cell wall, produced after the cell has stopped growing, also contains polysaccharides and is strengthened by polymeric lignin covalently cross-linked to hemicellulose. Cellulose is a homopolymer of anhydrocellobiose and thus a linear beta-(1-4)-D-glucan, while hemicelluloses include a variety of compounds, such as xylans, xyloglucans, arabinoxylans, and mannans in complex branched structures with a spectrum of substituents. Although generally polymorphous, cellulose is found in plant tissue primarily as an insoluble crystalline matrix of parallel glucan chains. Hemicelluloses usually hydrogen bond to cellulose, as well as to other hemicelluloses, which help stabilize the cell wall matrix.

Cellulose is generally found, for example, in the stems, leaves, hulls, husks, and cobs of plants or leaves, branches, and wood of trees. The cellulosic material can be, but is not limited to, agricultural residue, herbaceous material (including energy crops), municipal solid waste, pulp and paper mill residue, waste paper, and wood (including forestry residue) (see, for example, Wiselogel et al., 1995, in Handbook on Bioethanol (Charles E. Wyman, editor), pp. 105-118, Taylor & Francis, Washington D.C.; Wyman, 1994, Bioresource Technology 50: 3-16; Lynd, 1990, Applied Biochemistry and Biotechnology 24/25: 695-719; Mosier et al., 1999, Recent Progress in Bioconversion of Lignocellulosics, in Advances in Biochemical Engineering/Biotechnology, T. Scheper, managing editor, Volume 65, pp. 23-40, Springer-Verlag, New York). It is understood herein that the cellulose may be in the form of lignocellulose, a plant cell wall material containing lignin, cellulose, and hemicellulose in a mixed matrix. In a preferred aspect, the cellulosic material is any biomass material. In another preferred aspect, the cellulosic material is lignocellulose, which comprises cellulose, hemicelluloses, and lignin.

In one aspect, the cellulosic material is agricultural residue. In another aspect, the cellulosic material is herbaceous material (including energy crops). In another aspect, the cellulosic material is municipal solid waste. In another aspect, the cellulosic material is pulp and paper mill residue. In another aspect, the cellulosic material is waste paper. In another aspect, the cellulosic material is wood (including forestry residue).

In another aspect, the cellulosic material is arundo. In another aspect, the cellulosic material is bagasse. In another aspect, the cellulosic material is bamboo. In another aspect, the cellulosic material is corn cob. In another aspect, the cellulosic material is corn fiber. In another aspect, the cellulosic material is corn stover. In another aspect, the cellulosic material is miscanthus. In another aspect, the cellulosic material is orange peel. In another aspect, the cellulosic material is rice straw. In another aspect, the cellulosic material is switchgrass. In another aspect, the cellulosic material is wheat straw.

In another aspect, the cellulosic material is aspen. In another aspect, the cellulosic material is eucalyptus. In another aspect, the cellulosic material is fir. In another aspect, the cellulosic material is pine. In another aspect, the cellulosic material is poplar. In another aspect, the cellulosic material is spruce. In another aspect, the cellulosic material is willow.

In another aspect, the cellulosic material is algal cellulose. In another aspect, the cellulosic material is bacterial cellulose. In another aspect, the cellulosic material is cotton linter. In another aspect, the cellulosic material is filter paper. In another aspect, the cellulosic material is microcrystalline cellulose. In another aspect, the cellulosic material is phosphoric-acid treated cellulose.

In another aspect, the cellulosic material is an aquatic biomass. As used herein the term “aquatic biomass” means biomass produced in an aquatic environment by a photosynthesis process. The aquatic biomass can be algae, emergent plants, floating-leaf plants, or submerged plants.

The cellulosic material may be used as is or may be subjected to pretreatment, using conventional methods known in the art, as described herein. In a preferred aspect, the cellulosic material is pretreated.

Chitinolytic enzymes: The term “chitinolytic enzymes” means chitin-hydrolyzing enzymes, such as chitinase (EC 3.2.1.14) and chitobiase (beta-N-acetyl-D-glycosaminidase, EC 3.2.1.30).

Chitinous material: The term “chitinous material” means a material containing chitin which is a nitrogen-containing polysaccharide. Chitin is a major component of the exoskeletons of arthropods. Chitin consists of straight chains of N-acetyl-D-glucosamine residues linked beta-1,4. Chitin is also found in the walls of diatoms, fungi, and higher plants and usually occurs together with other polysaccharides, proteins, and/or inorganic salts

Endoglucanase: The term “endoglucanase” means an endo-1,4-(1,3; 1,4)-beta-D-glucan 4-glucanohydrolase (E.C. 3.2.1.4) that catalyzes endohydrolysis of 1,4-beta-D-glycosidic linkages in cellulose, cellulose derivatives (such as carboxymethyl cellulose and hydroxyethyl cellulose), lichenin, beta-1,4 bonds in mixed beta-1,3 glucans such as cereal beta-D-glucans or xyloglucans, and other plant material containing cellulosic components. Endoglucanase activity can be determined by measuring reduction in substrate viscosity or increase in reducing ends determined by a reducing sugar assay (Zhang et al., 2006, Biotechnology Advances 24: 452-481). For purposes of the present invention, endoglucanase activity is determined using carboxymethyl cellulose (CMC) as substrate according to the procedure of Ghose, 1987, Pure and Appl. Chem. 59: 257-268, at pH 5, 40° C.

Family 61 glycoside hydrolase: The term “Family 61 glycoside hydrolase” or “Family GH61” or “GH61” means a polypeptide falling into the glycoside hydrolase Family 61 according to Henrissat B., 1991, A classification of glycosyl hydrolases based on amino-acid sequence similarities, Biochem. J. 280: 309-316, and Henrissat B., and Bairoch A., 1996, Updating the sequence-based classification of glycosyl hydrolases, Biochem. J. 316: 695-696. The enzymes in this family were originally classified as a glycoside hydrolase family based on measurement of very weak endo-1,4-beta-D-glucanase activity in one family member. The structure and mode of action of these enzymes are non-canonical and they cannot be considered as bona fide glycosidases. However, they are kept in the CAZy classification on the basis of their capacity to enhance the breakdown of lignocellulose when used in conjunction with a cellulase or a mixture of cellulases.

Feruloyl esterase: The term “feruloyl esterase” means a 4-hydroxy-3-methoxycinnamoyl-sugar hydrolase (EC 3.1.1.73) that catalyzes the hydrolysis of 4-hydroxy-3-methoxycinnamoyl (feruloyl) groups from esterified sugar, which is usually arabinose in natural biomass substrates, to produce ferulate (4-hydroxy-3-methoxycinnamate). Feruloyl esterase is also known as ferulic acid esterase, hydroxycinnamoyl esterase, FAE-III, cinnamoyl ester hydrolase, FAEA, cinnAE, FAE-I, or FAE-II. For purposes of the present invention, feruloyl esterase activity is determined using 0.5 mM p-nitrophenylferulate as substrate in 50 mM sodium acetate pH 5.0. One unit of feruloyl esterase equals the amount of enzyme capable of releasing 1 μmole of p-nitrophenolate anion per minute at pH 5, 25° C.

Hemicellulolytic enzyme or hemicellulase: The term “hemicellulolytic enzyme” or “hemicellulase” means one or more (e.g., several) enzymes that hydrolyze a hemicellulosic material. See, for example, Shallom, D. and Shoham, Y. Microbial hemicellulases. Current Opinion In Microbiology, 2003, 6(3): 219-228). Hemicellulases are key components in the degradation of plant biomass. Examples of hemicellulases include, but are not limited to, an acetylmannan esterase, an acetylxylan esterase, an arabinanase, an arabinofuranosidase, a coumaric acid esterase, a feruloyl esterase, a galactosidase, a glucuronidase, a glucuronoyl esterase, a mannanase, a mannosidase, a pectin lyase, a xylanase, and a xylosidase. The substrates of these enzymes, the hemicelluloses, are a heterogeneous group of branched and linear polysaccharides that are bound via hydrogen bonds to the cellulose microfibrils in the plant cell wall, crosslinking them into a robust network. Hemicelluloses are also covalently attached to lignin, forming together with cellulose a highly complex structure. The variable structure and organization of hemicelluloses require the concerted action of many enzymes for its complete degradation. The catalytic modules of hemicellulases are either glycoside hydrolases (GHs) that hydrolyze glycosidic bonds, carbohydrate esterases (CEs), which hydrolyze ester linkages of acetate or ferulic acid side groups, or lyases, which undergo eliminative cleavage of α(1-4)-D-galacturonan methyl ester. These catalytic modules, based on homology of their primary sequence, can be assigned into GH and CE families. Some families, with an overall similar fold, can be further grouped into clans, marked alphabetically (e.g., GH-A). A most informative and updated classification of these and other carbohydrate active enzymes is available in the Carbohydrate-Active Enzymes (CAZy) database. Hemicellulolytic enzyme activities can be measured according to Ghose and Bisaria, 1987, Pure & Appl. Chem. 59: 1739-1752, at a suitable temperature, e.g., 50° C., 55° C., or 60° C., and pH, e.g., 5.0 or 5.5.

Hemicellulosic material: The term “hemicellulosic material” means a material comprising a class of plant cell wall polysaccharide that cannot be extracted from the wall by hot water or chelating agents, but can be extracted by aqueous alkali. Hemicellulosic material includes xylan, glucuronoxylan, arabinoxylan, arabinogalactan II, glucomannan, xyloglucan and galactomannan.

Polypeptide having cellulolytic enhancing activity: The term “polypeptide having cellulolytic enhancing activity” means a GH61 polypeptide that catalyzes the enhancement of the hydrolysis of a cellulosic material by enzyme having cellulolytic activity. For purposes of the present invention, cellulolytic enhancing activity is determined by measuring the increase in reducing sugars or the increase of the total of cellobiose and glucose from the hydrolysis of a cellulosic material by cellulolytic enzyme under the following conditions: 1-50 mg of total protein/g of cellulose in pretreated corn stover (PCS), wherein total protein is comprised of 50-99.5% w/w cellulolytic enzyme protein and 0.5-50% w/w protein of a GH61 polypeptide having cellulolytic enhancing activity for 1-7 days at a suitable temperature, such as 40° C.-80° C., e.g., 50° C., 55° C., 60° C., 65° C., or 70° C., and a suitable pH, such as 4-9, e.g., 5.0, 5.5, 6.0, 6.5, 7.0, 7.5, or 8.0, compared to a control hydrolysis with equal total protein loading without cellulolytic enhancing activity (1-50 mg of cellulolytic protein/g of cellulose in PCS). In a preferred aspect, a mixture of CELLUCLAST® 1.5 L (Novozymes NS, Bagærd, Denmark) in the presence of 2-3% of total protein weight Aspergillus oryzae beta-glucosidase (recombinantly produced in Aspergillus oryzae according to WO 02/095014) or 2-3% of total protein weight Aspergillus fumigatus beta-glucosidase (recombinantly produced in Aspergillus oryzae as described in WO 2002/095014) of cellulase protein loading is used as the source of the cellulolytic activity.

The GH61 polypeptides having cellulolytic enhancing activity enhance the hydrolysis of a cellulosic material catalyzed by enzyme having cellulolytic activity by reducing the amount of cellulolytic enzyme required to reach the same degree of hydrolysis preferably at least 1.01-fold, e.g., at least 1.05-fold, at least 1.10-fold, at least 1.25-fold, at least 1.5-fold, at least 2-fold, at least 3-fold, at least 4-fold, at least 5-fold, at least 10-fold, or at least 20-fold.

Pretreated corn stover: The term “PCS” or “Pretreated Corn Stover” means a cellulosic material derived from corn stover by treatment with heat and dilute sulfuric acid, alkaline pretreatment, neutral pretreatment, or any other pretreatment method known in the art

Starch material: The term “starch material” means any material comprising starch, which is a polysaccharide carbohydrate (C₆H₁₀O₅)ₙ consisting of a large number of glucose monosaccharide units joined together by alpha-1,4-glycosidic bonds or alpha-1,4-glycosidic bonds and alpha-1,6-glycosidic bonds. Starch is especially found in seeds, bulbs, and tubers. In the methods of the present invention, the starch material can be any material containing starch. Starch is generally obtained from the seeds of plants, such as corn, wheat, sorghum, or rice; and from the tubers and roots of plants such as cassava, potato, arrowroot, tapioca, and the pith of sago palm. Examples of starch materials suitable for use in the methods of the present invention include, but are not limited to, tubers, roots, stems, whole grains, corns, cobs, wheat, barley, rye, milo, sago, cassava, tapioca, sorghum, rice peas, beans, or sweet potatoes, or mixtures thereof, or cereals, sugar-containing raw materials, such as molasses, fruit materials, sugar cane or sugar beet, potatoes, and cellulosic materials, such as wood or plant residues, or mixtures thereof. Contemplated are both waxy and non-waxy types of corn and barley. The major commercial source of starch is corn, from which starch is extracted by wet milling processes. The starch material can also be lignocellulose containing starch.

In one aspect, the starch material is corn starch. In another aspect, the starch material is wheat starch. In another aspect, the starch material is sorghum starch. In another aspect, the starch material is rice starch. In another aspect, the starch material is cassava starch. In another aspect, the starch material is potato starch. In another aspect, the starch material is arrowroot starch. In another aspect, the starch material is tapioca starch. In another aspect, the starch material is sago palm starch.

The term “granular starch” means raw uncooked starch, i.e., starch in its natural form found in cereal, tubers or grains. Starch is formed within plant cells as tiny granules insoluble in water. When placed in cold water, the starch granules can absorb a small amount of the liquid and swell. At temperatures up to 50° C. to 75° C. the swelling can be reversible. However, with higher temperatures an irreversible swelling called “gelatinization” begins. Granular starch can be a highly refined starch, preferably at least 90%, more preferably at least 95%, more preferably at least 97%, and most preferably at least 99.5% pure, or it may be a more crude starch containing material comprising milled whole grain including non-starch fractions such as germ residues and fibers. The raw material, such as whole grain, is milled in order to open up the structure for further processing. Two milling processes are preferred: wet and dry milling. In dry milling whole kernels are milled and used. Wet milling gives a good separation of germ and meal (starch granules and protein) and is often applied at locations where starch hydrolysate is used in production of syrups. Both dry and wet-milling are well known in the art of starch processing.

The starch material may be reduced in particle size, preferably by dry or wet milling, in order to expose more surface area. In one aspect, the particle size is preferably between 0.05 to 3.0 mm, more preferably between 0.1 to 1.5 mm, and most preferably between 0.1 to 0.5 mm, so that preferably at least 30%, more preferably at least 50%, even more preferably at least 70%, and most preferably at least 90% of the starch material passes through a sieve with preferably a 0.05 to 3.0 mm screen, more preferably a 0.05 to 1.5 mm screen, and most preferably a 0.1 to 0.5 mm screen.

Xylan-containing material: The term “xylan-containing material” means any material comprising a plant cell wall polysaccharide containing a backbone of beta-(1-4)-linked xylose residues. Xylans of terrestrial plants are heteropolymers possessing a beta-(1-4)-D-xylopyranose backbone, which is branched by short carbohydrate chains. They comprise D-glucuronic acid or its 4-O-methyl ether, L-arabinose, and/or various oligosaccharides, composed of D-xylose, L-arabinose, D- or L-galactose, and D-glucose. Xylan-type polysaccharides can be divided into homoxylans and heteroxylans, which include glucuronoxylans, (arabino)glucuronoxylans, (glucurono)arabinoxylans, arabinoxylans, and complex heteroxylans. See, for example, Ebringerova et al., 2005, Adv. Polym. Sci. 186: 1-67.

In the methods of the present invention, any material containing xylan may be used. In a preferred aspect, the xylan-containing material is lignocellulose.

Xylan degrading activity or xylanolytic activity: The term “xylan degrading activity” or “xylanolytic activity” means a biological activity that hydrolyzes xylan-containing material. The two basic approaches for measuring xylanolytic activity include: (1) measuring the total xylanolytic activity, and (2) measuring the individual xylanolytic activities (e.g., endoxylanases, beta-xylosidases, arabinofuranosidases, alpha-glucuronidases, acetylxylan esterases, feruloyl esterases, and alpha-glucuronyl esterases). Recent progress in assays of xylanolytic enzymes was summarized in several publications including Biely and Puchard, 2006, Journal of the Science of Food and Agriculture 86(11): 1636-1647; Spanikova and Biely, 2006, FEBS Letters 580(19): 4597-4601; Herrmann et al., 1997, Biochemical Journal 321: 375-381.

Total xylan degrading activity can be measured by determining the reducing sugars formed from various types of xylan, including, for example, oat spelt, beechwood, and larchwood xylans, or by photometric determination of dyed xylan fragments released from various covalently dyed xylans. The most common total xylanolytic activity assay is based on production of reducing sugars from polymeric 4-O-methyl glucuronoxylan as described in Bailey, Biely, Poutanen, 1992, Interlaboratory testing of methods for assay of xylanase activity, Journal of Biotechnology 23(3): 257-270. Xylanase activity can also be determined with 0.2% AZCL-arabinoxylan as substrate in 0.01% TRITON® X-100 (4-(1,1,3,3-tetramethylbutyl)phenyl-polyethylene glycol) and 200 mM sodium phosphate buffer pH 6 at 37° C. One unit of xylanase activity is defined as 1.0 μmole of azurine produced per minute at 37° C., pH 6 from 0.2% AZCL-arabinoxylan as substrate in 200 mM sodium phosphate pH 6.

For purposes of the present invention, xylan degrading activity is determined by measuring the increase in hydrolysis of birchwood xylan (Sigma Chemical Co., Inc., St. Louis, Mo., USA) by xylan-degrading enzyme(s) under the following typical conditions: 1 ml reactions, 5 mg/ml substrate (total solids), 5 mg of xylanolytic protein/g of substrate, 50 mM sodium acetate pH 5, 50° C., 24 hours, sugar analysis using p-hydroxybenzoic acid hydrazide (PHBAH) assay as described by Lever, 1972, A new reaction for colorimetric determination of carbohydrates, Anal. Biochem 47: 273-279.

Xylanase: The term “xylanase” means a 1,4-beta-D-xylan-xylohydrolase (E.C. 3.2.1.8) that catalyzes the endohydrolysis of 1,4-beta-D-xylosidic linkages in xylans. For purposes of the present invention, xylanase activity is determined with 0.2% AZCL-arabinoxylan as substrate in 0.01% TRITON® X-100 and 200 mM sodium phosphate buffer pH 6 at 37° C. One unit of xylanase activity is defined as 1.0 μmole of azurine produced per minute at 37° C., pH 6 from 0.2% AZCL-arabinoxylan as substrate in 200 mM sodium phosphate pH 6.

### DETAILED DESCRIPTION OF THE INVENTION

The present invention relates to methods for analyzing degradation of a biomass material, comprising: (a) spreading a slurry of the biomass material over a multi-well fill plate to fill each well thereof, wherein each well of the multi-well fill plate comprises an aperture corresponding to a paired opening in the same well; (b) removing excess biomass material from the surfaces of the multi-well fill plate; (c) transferring the biomass material from the wells of the multi-well fill plate to corresponding wells of a multi-well reaction plate by a means that displaces the biomass material from the opening of each well of the multi-well fill plate into the corresponding opening of each well of the multi-well reaction plate, wherein the multi-well reaction plate comprises wells that are larger in volume and larger or equivalent in diameter or width compared to the multi-well fill plate; (d) adding an enzyme composition to each well of the multi-well reaction plate; (e) incubating the reaction plate for a period of time at a pH and a temperature for the enzyme composition to degrade the biomass material; and (f) detecting a signal resulting from the degradation of the biomass material, wherein an increase or a decrease in intensity of the signal indicates the amount of biomass material degraded by the enzyme composition.

The present disclosure describes methods for assaying, assessing, or quantifying the activity of an enzyme composition of interest at various dry weight percentages of a biomass material, including high dry weight percentages, and to methods for handling biomass materials, and to various tools for handling biomass materials or other slurried materials. The methods are useful for, inter alia, detecting soluble mono, di and polysaccharides and evaluating hydrolysis or degradation activity of enzyme compositions of interest. The present disclosure further describes an assay format that can be performed at high substrate content, for example, high dry weight percentage. The present invention further describes high- or medium-throughput assays, for example, a high-throughput assay for quantifying activity of an enzyme composition of interest toward a pretreated biomass material, e.g., pretreated corn stover (PCS).

In one aspect, the present disclosure provides methods of generating biomass material of high dry weight percentages, e.g., cellulosic material, for the hydrolysis or degradation of the biomass material, such as a lignocellulose hydrolysis, in multi-well microtiter or deep well plates in slurry form, including transfer of the material from a manufactured multi-well fill plate, e.g., a 96-well fill plate, to a multi-well reaction plate, e.g., a 96-well reaction plate. The transfer can be accomplished using a means of transfer including, but not limited to, centrifugation, gas pressure (e.g., air pressure), negative pressure (i.e., vacuum), physical displacement (e.g., by plunger or piston), or any other means known in the art.

The present invention provides that various biomass materials, e.g., cellulosic or starch materials, at high dry weight percentages can be accurately delivered into multi-well microtiter plates or multi-well deep plates using a volumetric approach, thereby creating the ability to screen and/or evaluate the performance of an enzyme composition of interest in degradation reactions, e.g., hydrolysis, containing the biomass material. Accordingly, the present invention makes it now possible to perform assays of an enzyme composition of interest at high dry weight percentages in high or medium throughput manner. Further, it is now possible to perform high-throughput assays for reduced periods of time. Moreover, it is now possible to detect residual biomass material and evaluate degradative activity of an enzyme composition of interest in a high-throughput format. The methods are also useful for discovering new enzymes, polypeptides, compositions or mixtures of enzymes, or synergizing activities with improved performance characteristics and/or quality control of commercial enzyme products. The methods of the present invention can eliminate the need to use HPLC and are faster by avoiding a typically lengthy HPLC assay as there are greater changes in viscosity, soluble sugar content, and the amount of residual solids in higher solids content assays, thereby permitting these parameters to be followed. In one aspect, the greater change in soluble reducing sugar and the high final concentration of reducing sugars allows easily automated or robotic colorimetric assays (e.g., glucose oxidase enzyme-coupled assay) to be used for quantification. In another aspect, the greater change in residual solids permits the extent of hydrolysis to be estimated or determined by visual inspection of the residual biomass pellet, or by quantification of change in size or volume of the residual biomass pellet.

In the methods of the present invention, a multi-well fill plate is used to transfer biomass material from the wells of the multi-well fill plate to corresponding wells of a multi-well reaction plate by a means that displaces the biomass material from the opening of each well of the multi-well fill plate into the corresponding opening of each well of the multi-well reaction plate. In one embodiment, the multi-well reaction plate comprises wells that are larger in volume and larger or equivalent in diameter or width compared to the multi-well fill plate to facilitate the transfer of the biomass to the multi-well reaction plate.

In another embodiment, the shape of the wells of the multi-well fill plate is conical, cylindrical, cubical, oval, rectangular, or spherical. In another embodiment, the shape of the wells is conical. In another embodiment, the shape of the wells is cylindrical. In another embodiment, the shape of the wells is cubical. In another embodiment, the shape of the wells is oval. In another embodiment, the shape of the wells is rectangular. In another embodiment, the shape of the wells is spherical. The wells of the multi-cell fill plate can be of any shape useful in practicing the methods of the present invention.

In another embodiment, the volume of each well of the multi-well fill plate is at least 1 μl, e.g., at least 5 μl, at least 10 μl, at least 20 μl, at least 30 μl, at least 40 μl, at least 50 μl, at least 60 μl, at least 70 μl, at least 80 μl, at least 90 μl, at least 100 μl, at least 125 μl, at least 150 μl, at least 175 μl, at least 200 μl, at least 250 μl, at least 500 μl, at least 750 μl, at least 1 ml, at least 2 ml, at least 5 ml, at least 10 ml, at least 25 ml, or at least 50 ml.

In another embodiment, the aperture corresponding to a paired opening of each well in the multi-well fill plate is the same size as the opening of each well. In another embodiment, the aperture corresponding to a paired opening of each well in the multi-well fill plate is smaller in size than the opening of each well. In another embodiment, the aperture corresponding to a paired opening in the multi-well fill plate is a pin hole. In another embodiment, the aperture corresponding to a paired opening in the multi-well fill plate is at least 0.1-fold, at least 0.25-fold, at least 0.5-fold, at least 0.75-fold, at least 1-fold, at least 2-fold, at least 5-fold, at least 10-fold smaller, at least 20-fold, at least 30-fold at least 40-fold, at least 50-fold, at least 60-fold, at least 70-fold, at least 80-fold, or at least 90-fold smaller than the paired opening in the multi-well fill plate.

In another embodiment, the multi-well fill plate comprises wells that correspond to wells of a multi-well reaction plate. The wells of the multi-well reaction plate may or may not comprise additional apertures extending through the depth of the plate and open to the opposite side. In a preferred aspect, the wells of the multi-well reaction plate contain only one opening.

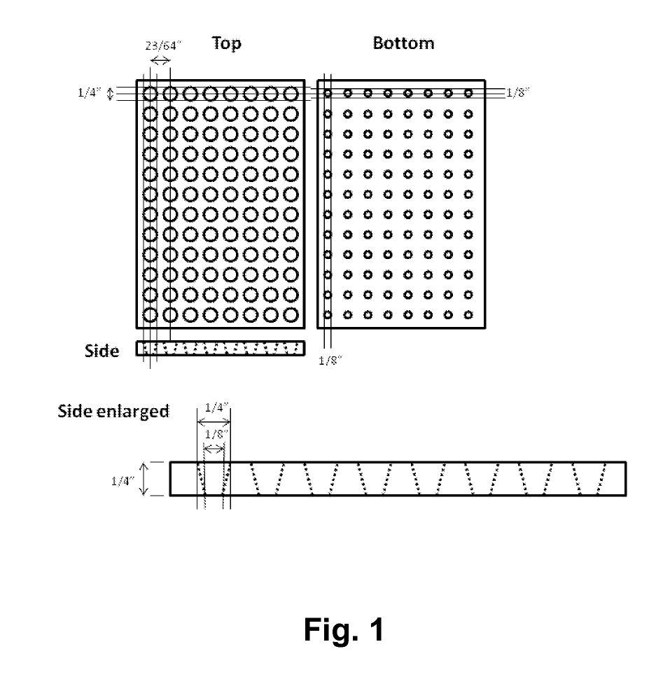

A multi-well fill plate can be manufactured using any suitable material, e.g., glass, metal (e.g., aluminum), plastic, TEFLON®, combinations thereof, etc. By way of illustration, a 96-well fill plate can be manufactured by machining an aluminum plate of depth ¼ inch with 96, cone-shaped wells, diameter ¼ inch at the upper surface and diameter ⅛ inch at the lower surface. The center of each well is at an equivalent position to the center of a corresponding well in a standard 96-well microtiter plate, approximately 23/64 inch on center. The resulting volume of each well is approximately 135 μl. A schematic diagram of a representative multi-well fill plate is shown in FIG. 1. In a preferred embodiment, the number of wells in the multi-well fill plate and the corresponding multi-well reaction plate is maximized, and the volume minimized to permit higher throughput. In another preferred embodiment, the multi-well fill plate is composed of TEFLON®. In another preferred embodiment, the multi-well fill plate has raised edges or clips to facilitate alignment of the multi-well fill plate with the multi-well reaction plate.

In the methods of the present invention, a multi-well reaction plate is used for performing the degradation or hydrolysis of the biomass material. The multi-well reaction plate can be a standard multi-well microtiter plate or multi-well deep well plate, which is available from many commercial vendors. Alternatively, the multi-well reaction plate can be manufactured using specifications in accordance with the present invention.

In the methods of the present invention, a slurry of the biomass material is spread over the multi-well fill plate to fill each well of the plate. The spreading of the slurry of the biomass material over the multi-well fill plate can be performed manually, robotically, or mechanically. For example, the spreading can be accomplished using a gang-punch, a microplate punch, a press, a razor blade, a spatula, a wiper, or related implement. The implement can be composed of glass, metal, rubber, plastic, TEFLON®, or wood, or combinations thereof, or can be composed of another material. The spreading can also be accomplished by screening small particles through a filter, sieve, or screen onto the multi-well fill plate. The spreading can also be accomplished by pouring loose particles and settling, optionally while vibrating the multi-well fill plate. Excess biomass material is removed from the surfaces of the multi-well fill plate. Removing the excess biomass can be performed manually, robotically, or mechanically.

In one embodiment, the slurry is an aqueous slurry. In another embodiment, the slurry is a powder. In another embodiment, the slurry is a non-aqueous slurry. In another embodiment, the slurry is an ionic liquid slurry. In another embodiment, the slurry is an aqueous-ethanol slurry (e.g., 40-60% ethanol).

In one embodiment, the biomass slurry is at least 1% total solids, e.g., at least 5% total solids, at least 10% total solids, at least 15% total solids, at least 20% total solids, at least 25% total solids, at least 30% total solids, at least 35% total solids, at least 40% total solids, at least 45% total solids, at least 50% total solids, at least 55% total solids, at least 60% total solids, at least 65% total solids, at least 70% total solids, at least 75% total solids, at least 80% total solids, at least 85% total solids, at least 90% total solids, or at least 95% total solids.

In another embodiment, the biomass slurry contains one or more (e.g., several) internal standards for normalizing differences of the biomass material between the wells. Any suitable standard can be used. Examples of such standards include, but are not limited to, acetate, citrate, or aldol sugars.

In the methods of the present invention, the biomass material is transferred from the wells of the multi-well fill plate to corresponding wells of a multi-well reaction plate by a means that displaces the biomass material from the opening of each well of the multi-well fill plate into the corresponding opening of each well of the multi-well reaction plate. The means that displaces the biomass material from the multi-well fill plate into the multi-well reaction plate can be centrifugation. The means can be pressured gas, e.g., pressurized air. The means can be physical displacement, e.g., single or multiple plungers or pins. The means can be a microplate punching device. The means can be vacuum or negative pressure, with or without a gasket or manifold. The means can also be a combination of any of the above means. Any means known in the art can be used.

In one embodiment, the volume of each well of the multi-well reaction plate is at least 1 μl, e.g., at least 5 μl, at least 10 μl, at least 20 μl, at least 30 μl, at least 40 μl, at least 50 μl, at least 60 μl, at least 70 μl, at least 80 μl, at least 90 μl, at least 100 μl, at least 125 μl, at least 150 μl, at least 175 μl, at least 200 μl, at least 250 μl, at least 500 μl, at least 750 μl, at least 1 ml, at least 2 ml, at least 5 ml, at least 10 ml, at least 25 ml, or at least 50 ml.

In another embodiment, the multi-well reaction plate comprises wells that are larger in volume and larger or equivalent in diameter or width compared to the wells of the multi-well fill plate to facilitate complete transfer of the biomass material. In another embodiment, the volume and diameter or width of each well of the multi-well reaction plate is at least 5%, e.g., at least 10%, at least 20%, at least 30%, at least 40%, at least 50%, at least 100%, at least 500%, at least 1000%, or at least 5000% larger than the volume and diameter or width of each well of the multi-well fill plate.

In another embodiment, the shape of the wells of the multi-well reaction plate is conical, cylindrical, cubical, oval, rectangular, or spherical. In another embodiment, the shape of the wells is conical. In another embodiment, the shape of the wells is cylindrical. In another embodiment, the shape of the wells is cubical. In another embodiment, the shape of the wells is oval. In another embodiment, the shape of the wells is rectangular. In another embodiment, the shape of the wells is spherical. The wells of the multi-cell reaction plate can be of any shape useful in practicing the methods of the present invention.

In another aspect, the present disclosure provides methods of mixing high solids reactions in multi-well plates, e.g., 96-well plates, thoroughly before and during hydrolysis. Multi-well reactions, e.g., 96-well, plate format reactions, can be mixed by inverting plates and centrifuging, re-inverting the plates and centrifuging, and repeating for one or more (i.e., several) repetitions. High solids hydrolysis or degradation reactions can be mixed continuously during the reaction by addition of a means of mixing into each well, and placement of the multi-well reaction plate in a shaking incubator, long axis of the well parallel to the shaking axis of the incubator. The means of mixing can be added into each well of a multi-well reaction plate by means of a multi-well, adhesive-backed sheet. In one embodiment, the means of mixing is selected from the group consisting of a bead (e.g., glass bead) or a stir bar. In another embodiment, the means of mixing is selected from the group consisting of rotation, inversion, vibration, and agitation. In another embodiment, the means of mixing is a combination of a bead (e.g., glass bead) or a stir bar with rotation, inversion, vibration, or agitation. Any means of mixing known in the art can be used.

In another aspect, the present disclosure provides methods of varying the biomass material content, e.g., cellulose content, of reactions in multi-well reaction plates, e.g., 96-well plates, by altering the final volume of the reaction of the high solids reactions. The included volume of each well of the fill plate is measured or known beforehand; therefore the volume of the biomass material added to each well of the reaction plate used for high solids hydrolysis or degradation is similarly known. In embodiments, measuring the total mass of the multi-well fill plate before and after transfer of the biomass material yields the total mass of biomass in the reaction plate; thus the average mass per well can be determined. Addition of suitable volume or mass of reaction components, i.e., buffer and/or water and/or enzyme composition of interest is then performed to generate the final solids content desired.

In the methods of the present invention, the desired final solids content and the solids content of the original biomass slurry inform the limits of the volume of enzyme and buffer that can be added. The volume or mass of enzyme composition, buffer, additional compounds being investigated, and water added to each well are made to correspond to the volume or mass necessary to dilute the biomass slurry to the desired final solids content. In aspects, the volume or mass of enzyme composition added corresponds to the final concentration of enzyme desired and the final reaction volume, determined as the sum of the volume of biomass slurry added to the well and the total volume of other components added. In aspects, buffer or other compounds under study are added to each well as concentrated stocks at volumes corresponding to the desired final concentration in the final reaction volume (e.g., 1/10 reaction volume for a 10-fold concentrated stock) familiar to one skilled in the art. In aspects, water or the desired reaction solvent is added to each well in either the volume or the mass necessary for the total volume or mass of all components added to each well to sum to the desired final volume or mass. In other aspects, enzyme composition, buffer, and all other non-slurry components are premixed in one or more (e.g., several) solutions at concentrations higher than the final desired concentrations in a manner that accounts for the dilution that occurs upon addition to the biomass slurry in each well.

In the methods of the present invention, an enzyme composition of interest is added to each well of the multi-well reaction plate and the reaction plate is incubated for a period of time at a temperature and pH for the enzyme composition to degrade or hydrolyze the biomass material.

The hydrolysis or degradation reaction can last up to 200 hours, but is typically performed for about 12 to about 120 hours, e.g., about 16 to about 72 hours or about 24 to about 48 hours. The temperature can be in the range of about 25° C. to about 70° C., e.g., about 30° C. to about 65° C., about 40° C. to about 60° C., or about 50° C. to about 60° C. The pH can be in the range of about 3 to about 9, e.g., about 3.5 to about 7, about 4 to about 6, or about 4.5 to about 5.5. However, any period of time, temperature, or pH can be used in the methods of the present invention.

In the methods of the present invention, a signal is detected resulting from the degradation of the biomass material, wherein an increase or a decrease in intensity of the signal indicates the amount of biomass material degraded by the enzyme composition. The signal detected can be selected from the group consisting of reducing sugars (e.g., glucose and/or xylose), disappearance of biomass material, fluidity of the biomass material, viscosity, and absorbance. Any detectable signal known in the art can be used.

In another aspect, the present disclosure provides methods of determining whether an enzyme of interest or polypeptide of interest affects degradation or hydrolysis of a biomass material, by generating high total solids content of a biomass material in a multi-well microtiter or deep well plate by transfer of the biomass material from a manufactured multi-well fill plate to a multi-well reaction plate by centrifugation or other means. In an embodiment, the methods comprise (a) spreading the biomass material over a multi-well fill plate thereby filling each well, removing excess biomass material, and transferring the biomass contained in each well to a corresponding well of a multi-well reaction plate, (b) varying the high dry weight percentage of the reaction mixtures as desired, (c) adding an enzyme of interest and/or a polypeptide of interest and/or an accessory enzyme and/or mixtures thereof to the biomass material, (d) mixing the reaction wells as desired, and (e) determining the extent of hydrolysis or degradation in each reaction well.

In another aspect, the present disclosure provides a high-throughput method of analyzing the effects of an enzyme, a polypeptide, a chemical compound, an enzyme composition, or a combination thereof on biomass hydrolysis or degradation and/or activity of other enzymes. High-throughput methods are described by, for example, Michael Lamsa, Nils Buchberg Jensen, and Steen Krogsgaard, Screen Automation and Robotics, in Enzyme Functionality: Design, Engineering, and Screening, A. Svendsen, editor, Marcel Dekker, 2003. As the amount of biomass material, e.g., lignocellulose, can be high in the assays, the amount of a component of the biomass material, e.g., cellulose, is correspondingly high. The extent of hydrolysis by an enzyme, a polypeptide, a chemical compound, an enzyme composition, or a combination thereof can be assayed by examination of the volume of residual biomass material. The residual biomass material can be transferred to a conical bottomed plate and the radius of the pellet measured by scanning and image analysis. In one embodiment, the residual biomass material is pelleted by centrifugation and assayed by inspection. In another embodiment, the drop in viscosity concomitant to hydrolysis or degradation of high solids is used to analyze the effects of an enzyme, a polypeptide, a chemical compound, an enzyme composition, or a combination thereof. In another embodiment, the multi-well plate is simply rotated, and the fluidity of the wells can be used to determine the relative effects by inspection. In other embodiments, the extent of degradation or hydrolysis can be determined by HPLC, UHPLC, IC, LC-MS, UHPLC-MS, or NIR analysis of soluble mono, di and oligosaccharides.

In another aspect, the present disclosure provides methods of analyzing the performance of an enzyme, an accessory enzyme, a polypeptide, a chemical compound, an enzyme composition, or a combination thereof by generating high total solids content of a biomass material in a multi-well reaction plate by transfer of the biomass material from a multi-well fill plate to the multi-well reaction plate by centrifugation or other means. In an embodiment, the methods include (a) spreading biomass material over a multi-well fill thereby filling each well, removing excess biomass and transferring the biomass contained in each well to a corresponding well of a multi-well reaction plate, (b) varying the high dry weight percentage of the reaction mixtures as desired, (c) adding an enzyme, an accessory enzyme, a polypeptide, a chemical compound, an enzyme composition, or a combination thereof to the biomass material, (d) mixing the reaction wells as desired, and (e) determining the extent of hydrolysis or degradation in each reaction well. Non-limiting examples of enzyme performance include an indication of thermostability, thermal activity, pH stability, pH activity, resistance to or sensitivity to inhibition, capacity to enhance or inhibit enzymatic activity of one or more (i.e., several) other enzymes, activity in the presence of various salts, phenolics and other compounds typically found in biomass slurries, and, importantly, sensitivity to dry weight percentage (i.e., solids content).

The methods of the present invention may be used to analyze hydrolysis or degradation of a biomass material by an enzyme composition, by generating high total content in a multi-well reaction plate by transfer of the biomass material from a multi-well fill plate to the multi-well reaction plate by centrifugation or other means. In an embodiment, the methods include (a) spreading lignocellulosic biomass over a multi-well fill plate thereby filling each well, (b) removing excess biomass and transferring the biomass contained in each well to a corresponding well of the multi-well reaction plate, (c) varying the high dry weight percentage of the reaction mixtures as desired, (d) adding an enzyme composition to the biomass material, (e) mixing the reaction wells as desired, and (f) determining the extent of hydrolysis or degradation in each reaction well.

The methods of the present invention may be used to determine whether an enzyme or polypeptide of interest affects hydrolysis or degradation of a biomass material by an enzyme composition by generating a high dry weight percentage in a multi-well microtiter or deep well reaction plate by transfer of the biomass material from a multi-well fill plate to a multi-well reaction plate by centrifugation or other means. In an embodiment, the methods comprise (a) spreading the biomass material over a multi-well fill plate thereby filling each well, removing excess biomass material and transferring the biomass material contained in each well of the fill plate to a corresponding well of a multi-well reaction plate, (b) varying the high dry weight percentage of the reaction mixtures as desired, (c) adding an enzyme composition of interest in the presence of an enzyme or polypeptide of interest to the biomass material, (d) mixing the reaction wells as desired, (e) determining the extent of hydrolysis in each reaction well, and (f) determining the extent of hydrolysis in each reaction well. It can be determined whether the extent of hydrolysis is altered, indicating activity of the enzyme or polypeptide of interest and/or change in the amount of biomass hydrolyzed by the enzyme of interest.

Non-limiting examples of suitable enzymes of interest in each of the aspects above include enzyme selected from the group consisting of: (a) wild-type exo-cellobiohydrolase, wild-type endo-1,4-β-glucanase, wild-type exo-1,4-β-glucosidase, wild-type cellobiase, wild-type GH61 polypeptide, or combinations thereof; and (b) variant exo-cellobiohydrolase, variant endo-1,4β-glucanase, variant exo-1,4β-glucosidase, variant cellobiase, variant GH61 polypeptide, or combinations thereof. Combinations of these wild-types and variants are also contemplated as enzyme mixtures of interest in accordance with the present disclosure. Fragments having the activity of (a) or (b) are also contemplated herein. Various compositions are described herein.

In another aspect, the present disclosure provides a system for evaluating enzyme and/or polypeptide performance by providing a system for automated filling of plates with biomass material. In one embodiment, a robotized or mechanical spreader is used to fill multi-well fill plates with the biomass material. In another embodiment, a robotized mechanical or hydraulic press is used to fill the wells of the multi-well fill plates with biomass material. In another embodiment, pressurized air or other gas is used to fill the wells of the multi-well fill plates with biomass material. In another embodiment, pressurized air or other gas is used to transfer the biomass from the multi-well fill plate to the multi-well reaction plate. In another embodiment, transfer of mixing means to the multi-well plate is performed by robotic or automated means. In another embodiment pipetting an enzyme composition and/or water and/or buffer is performed by robotic or automated means. In another embodiment, dilution and or sampling to obtain a signal indicating hydrolysis is performed by robotic or automated means.

In another aspect, the present disclosure provides methods of grinding a biomass material, e.g., lignocellulose, to facilitate their use in the above described aspects of the present disclosure where necessary, or wherein grinding improves the performance of the assay. In one embodiment, the biomass material is ground using a wet mill. In an embodiment, the biomass material is ground using a ball mill. In another embodiment, the biomass material is milled using an attritor mill.

In another aspect, the present disclosure provides quality control methods. For example, quality control parameters such as polypeptide activity, enzyme activity, e.g., cellulase activity or cellulolytic enhancing activity, and/or stability can be checked by a manufacturer after one or more batches of an enzyme or a polypeptide are produced. Further, quality control parameters such as polypeptide activity, enzyme activity, and/or stability can also be checked by a purchaser of an enzyme or polypeptide.

In another aspect, the biomass material is pretreated, as described herein, before use in the methods of the present invention.

In another aspect, the methods further comprise fermenting the hydrolysed or degraded biomass material with one or more (e.g., several) fermenting microorganisms to produce a fermentation product, as described herein. The fermentation product may be measured using standard methods in the art.

Non-limiting examples of biomass materials in each of the aspects above are cellulosic material, hemicellulosic material, starch material, and combinations thereof, and chitinous material. In one embodiment, the biomass material is cellulosic material. In another embodiment, the biomass material is hemicellulosic material. In another embodiment, the biomass material is starch material. In another embodiment, the biomass material is chitinous material.

The present invention also relates to a multi-well fill plate for transferring a slurry of a biomass material to a multi-well reaction plate, wherein each well of the multi-well fill plate comprises an aperture corresponding to a paired opening in the same well.

### Compositions

For cellulosic and hemicellulosic materials, the enzyme composition can comprise any protein useful in degrading the cellulosic or hemicellulosic material. In one aspect, the enzyme composition comprises or further comprises one or more (e.g., several) proteins selected from the group consisting of a cellulase, a GH61 polypeptide having cellulolytic enhancing activity, a hemicellulase, an esterase, an expansin, a laccase, a ligninolytic enzyme, a pectinase, a peroxidase, a protease, and a swollenin. In another aspect, the cellulase is preferably one or more (e.g., several) enzymes selected from the group consisting of an endoglucanase, a cellobiohydrolase, and a beta-glucosidase. In another aspect, the hemicellulase is preferably one or more (e.g., several) enzymes selected from the group consisting of an acetylmannan esterase, an acetylxylan esterase, an arabinanase, an arabinofuranosidase, a coumaric acid esterase, a feruloyl esterase, a galactosidase, a glucuronidase, a glucuronoyl esterase, a mannanase, a mannosidase, a pectin lyase, a xylanase, and a xylosidase.

In another aspect, the enzyme composition comprises one or more (e.g., several) cellulolytic enzymes. In another aspect, the enzyme composition comprises or further comprises one or more (e.g., several) hemicellulolytic enzymes. In another aspect, the enzyme composition comprises one or more (e.g., several) cellulolytic enzymes and one or more (e.g., several) hemicellulolytic enzymes. In another aspect, the enzyme composition comprises one or more (e.g., several) enzymes selected from the group of cellulolytic enzymes and hemicellulolytic enzymes. In another aspect, the enzyme composition comprises an endoglucanase. In another aspect, the enzyme composition comprises a cellobiohydrolase. In another aspect, the enzyme composition comprises a beta-glucosidase. In another aspect, the enzyme composition comprises a polypeptide having cellulolytic enhancing activity. In another aspect, the enzyme composition comprises an endoglucanase and a polypeptide having cellulolytic enhancing activity. In another aspect, the enzyme composition comprises a cellobiohydrolase and a polypeptide having cellulolytic enhancing activity. In another aspect, the enzyme composition comprises a beta-glucosidase and a polypeptide having cellulolytic enhancing activity. In another aspect, the enzyme composition comprises an endoglucanase and a cellobiohydrolase. In another aspect, the enzyme composition comprises an endoglucanase and a beta-glucosidase. In another aspect, the enzyme composition comprises a cellobiohydrolase and a beta-glucosidase. In another aspect, the enzyme composition comprises an endoglucanase, a cellobiohydrolase, and a polypeptide having cellulolytic enhancing activity. In another aspect, the enzyme composition comprises an endoglucanase, a beta-glucosidase, and a polypeptide having cellulolytic enhancing activity. In another aspect, the enzyme composition comprises a cellobiohydrolase, a beta-glucosidase, and a polypeptide having cellulolytic enhancing activity. In another aspect, the enzyme composition comprises an endoglucanase, a cellobiohydrolase, and a beta-glucosidase. In another aspect, the enzyme composition comprises an endoglucanase, a cellobiohydrolase, a beta-glucosidase, and a polypeptide having cellulolytic enhancing activity.

In another aspect, the enzyme composition comprises an acetylmannan esterase. In another aspect, the enzyme composition comprises an acetylxylan esterase. In another aspect, the enzyme composition comprises an arabinanase (e.g., alpha-L-arabinanase). In another aspect, the enzyme composition comprises an arabinofuranosidase (e.g., alpha-L-arabinofuranosidase). In another aspect, the enzyme composition comprises a coumaric acid esterase. In another aspect, the enzyme composition comprises a feruloyl esterase. In another aspect, the enzyme composition comprises a galactosidase (e.g., alpha-galactosidase and/or beta-galactosidase). In another aspect, the enzyme composition comprises a glucuronidase (e.g., alpha-D-glucuronidase). In another aspect, the enzyme composition comprises a glucuronoyl esterase. In another aspect, the enzyme composition comprises a mannanase. In another aspect, the enzyme composition comprises a mannosidase (e.g., beta-mannosidase). In another aspect, the enzyme composition comprises a xylanase. In a preferred aspect, the xylanase is a Family 10 xylanase. In another aspect, the enzyme composition comprises a xylosidase (e.g., beta-xylosidase).

In another aspect, the enzyme composition comprises an esterase. In another aspect, the enzyme composition comprises an expansin. In another aspect, the enzyme composition comprises a laccase. In another aspect, the enzyme composition comprises a ligninolytic enzyme. In a preferred aspect, the ligninolytic enzyme is a manganese peroxidase. In another preferred aspect, the ligninolytic enzyme is a lignin peroxidase. In another preferred aspect, the ligninolytic enzyme is a H₂O₂-producing enzyme. In another aspect, the enzyme composition comprises a pectinase. In another aspect, the enzyme composition comprises a peroxidase. In another aspect, the enzyme composition comprises a protease. In another aspect, the enzyme composition comprises a swollenin.

One or more (e.g., several) components of the enzyme composition may be wild-type proteins, recombinant proteins, or a combination of wild-type proteins and recombinant proteins. For example, one or more (e.g., several) components may be native proteins of a cell, which is used as a host cell to express recombinantly one or more (e.g., several) other components of the enzyme composition. One or more (e.g., several) components of the enzyme composition may be produced as monocomponents, which are then combined to form the enzyme composition. The enzyme composition may be a combination of multicomponent and monocomponent protein preparations.

The enzymes used in the methods of the present invention may be in any form suitable for use, such as, for example, a whole fermentation broth formulation or a cell composition (WO 90/15861 or WO 2010/096673), a cell lysate with or without cellular debris, a semi-purified or purified enzyme preparation, or a host cell as a source of the enzymes. The enzyme composition may be a dry powder or granulate, a non-dusting granulate, a liquid, a stabilized liquid, or a stabilized protected enzyme. Liquid enzyme preparations may, for instance, be stabilized by adding stabilizers such as a sugar, a sugar alcohol or another polyol, and/or lactic acid or another organic acid according to established processes.

The optimum amounts of the cellulolytic or hemicellulolytic enzyme and polypeptides having enzyme activity of interest depend on several factors including, but not limited to, the mixture of component cellulolytic enzymes and/or hemicellulolytic enzymes, the cellulosic material, the concentration of cellulosic material, the pretreatment(s) of the cellulosic material, temperature, time, pH, and inclusion of fermenting organism (e.g., yeast for Simultaneous Saccharification and Fermentation).

In one aspect, an effective amount of cellulolytic or hemicellulolytic enzyme to the cellulosic material is about 0.5 to about 50 mg, e.g., about 0.5 to about 40 mg, about 0.5 to about 25 mg, about 0.75 to about 20 mg, about 0.75 to about 15 mg, about 0.5 to about 10 mg, or about 2.5 to about 10 mg per g of the cellulosic material.

In another aspect, an effective amount of a polypeptide having enzyme activity of interest to the cellulosic material is about 0.01 to about 50.0 mg, e.g., about 0.01 to about 40 mg, about 0.01 to about 30 mg, about 0.01 to about 20 mg, about 0.01 to about 10 mg, about 0.01 to about 5 mg, about 0.025 to about 1.5 mg, about 0.05 to about 1.25 mg, about 0.075 to about 1.25 mg, about 0.1 to about 1.25 mg, about 0.15 to about 1.25 mg, or about 0.25 to about 1.0 mg per g of the cellulosic material.

In another aspect, an effective amount of a polypeptide having enzyme activity of interest to cellulolytic or hemicellulolytic enzyme is about 0.005 to about 1.0 g, e.g., about 0.01 to about 1.0 g, about 0.15 to about 0.75 g, about 0.15 to about 0.5 g, about 0.1 to about 0.5 g, about 0.1 to about 0.25 g, or about 0.05 to about 0.2 g per g of cellulolytic or hemicellulolytic enzyme.

The cellulolytic or hemicellulolytic enzyme and polypeptides having enzyme activity of interest as well as other proteins/polypeptides useful in the degradation of the cellulosic material, e.g., GH61 polypeptides having cellulolytic enhancing activity (collectively hereinafter “polypeptides having enzyme activity”) can be derived or obtained from any suitable origin, including, bacterial, fungal, yeast, plant, or mammalian origin. The term “obtained” also means herein that the enzyme may have been produced recombinantly in a host organism employing methods described herein, wherein the recombinantly produced enzyme is either native or foreign to the host organism or has a modified amino acid sequence, e.g., having one or more (e.g., several) amino acids that are deleted, inserted and/or substituted, i.e., a recombinantly produced enzyme that is a mutant and/or a fragment of a native amino acid sequence or an enzyme produced by nucleic acid shuffling processes known in the art. Encompassed within the meaning of a native enzyme are natural variants and within the meaning of a foreign enzyme are variants obtained recombinantly, such as by site-directed mutagenesis or shuffling.

A polypeptide having enzyme activity may be a bacterial polypeptide. For example, the polypeptide may be a Gram-positive bacterial polypeptide such as a Bacillus, Streptococcus, Streptomyces, Staphylococcus, Enterococcus, Lactobacillus, Lactococcus, Clostridium, Geobacillus, Caldicellulosiruptor, Acidothermus, Thermobifidia, or Oceanobacillus polypeptide having enzyme activity, or a Gram-negative bacterial polypeptide such as an E. coli, Pseudomonas, Salmonella, Campylobacter, Helicobacter, Flavobacterium, Fusobacterium, Ilyobacter, Neisseria, or Ureaplasma polypeptide having enzyme activity.

In one aspect, the polypeptide is a Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, or Bacillus thuringiensis polypeptide having enzyme activity.

In another aspect, the polypeptide is a Streptococcus equisimilis, Streptococcus pyogenes, Streptococcus uberis, or Streptococcus equi subsp. Zooepidemicus polypeptide having enzyme activity.

In another aspect, the polypeptide is a Streptomyces achromogenes, Streptomyces avermitilis, Streptomyces coelicolor, Streptomyces griseus, or Streptomyces lividans polypeptide having enzyme activity.

The polypeptide having enzyme activity may also be a fungal polypeptide, and more preferably a yeast polypeptide such as a Candida, Kluyveromyces, Pichia, Saccharomyces, Schizosaccharomyces, or Yarrowia polypeptide having enzyme activity; or more preferably a filamentous fungal polypeptide such as an Acremonium, Agaricus, Alternaria, Aspergillus, Aureobasidium, Botryosphaeria, Ceriporiopsis, Chaetomidium, Chrysosporium, Claviceps, Cochliobolus, Coprinopsis, Coptotermes, Corynascus, Cryphonectria, Cryptococcus, Diplodia, Exidia, Filibasidium, Fusarium, Gibberella, Holomastigotoides, Humicola, Irpex, Lentinula, Leptospaeria, Magnaporthe, Melanocarpus, Meripilus, Mucor, Myceliophthora, Neocallimastix, Neurospora, Paecilomyces, Penicillium, Phanerochaete, Piromyces, Poitrasia, Pseudoplectania, Pseudotrichonympha, Rhizomucor, Schizophyllum, Scytalidium, Talaromyces, Thermoascus, Thielavia, Tolypocladium, Trichoderma, Trichophaea, Verticillium, Volvariella, or Xylaria polypeptide having enzyme activity.

In one aspect, the polypeptide is a Saccharomyces carlsbergensis, Saccharomyces cerevisiae, Saccharomyces diastaticus, Saccharomyces douglasfi, Saccharomyces kluyveri, Saccharomyces norbensis, or Saccharomyces oviformis polypeptide having enzyme activity.

In another aspect, the polypeptide is an Acremonium cellulolyticus, Aspergillus aculeatus, Aspergillus awamori, Aspergillus fumigatus, Aspergillus foetidus, Aspergillus japonicus, Aspergillus nidulans, Aspergillus niger, Aspergillus oryzae, Chrysosporium keratinophilum, Chrysosporium lucknowense, Chrysosporium tropicum, Chrysosporium merdarium, Chrysosporium inops, Chrysosporium pannicola, Chrysosporium queenslandicum, Chrysosporium zonatum, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola grisea, Humicola insolens, Humicola lanuginosa, Irpex lacteus, Mucor miehei, Myceliophthora thermophila, Neurospora crassa, Penicillium funiculosum, Penicillium purpurogenum, Phanerochaete chrysosporium, Thielavia achromatica, Thielavia albomyces, Thielavia albopilosa, Thielavia australeinsis, Thielavia fimeti, Thielavia microspora, Thielavia ovispora, Thielavia peruviana, Thielavia spededonium, Thielavia setosa, Thielavia subthermophila, Thielavia terrestris, Trichoderma harzianum, Trichoderma koningii, Trichoderma longibrachiatum, Trichoderma reesei, Trichoderma viride, or Trichophaea saccata polypeptide having enzyme activity.

Chemically modified or protein engineered mutants of polypeptides having enzyme activity may also be used.

One or more (e.g., several) components of the enzyme composition may be a recombinant component, i.e., produced by cloning of a DNA sequence encoding the single component and subsequent cell transformed with the DNA sequence and expressed in a host (see, for example, WO 91/17243 and WO 91/17244). The host is preferably a heterologous host (enzyme is foreign to host), but the host may under certain conditions also be a homologous host (enzyme is native to host). Monocomponent cellulolytic proteins may also be prepared by purifying such a protein from a fermentation broth.

In one aspect, the one or more (e.g., several) cellulolytic enzymes comprise a commercial cellulolytic enzyme preparation. Examples of commercial cellulolytic enzyme preparations suitable for use in the present invention include, for example, CELLIC® CTec (Novozymes NS), CELLIC® CTec2 (Novozymes NS), CELLIC® CTec3 (Novozymes NS), CELLUCLAST™ (Novozymes NS), NOVOZYM™ 188 (Novozymes NS), CELLUZYME™ (Novozymes NS), CEREFLO™ (Novozymes NS), and ULTRAFLO™ (Novozymes NS), ACCELERASE™ (Genencor Int.), LAMINEX™ (Genencor Int.), SPEZYME™ CP (Genencor Int.), FILTRASE® NL (DSM); METHAPLUS® S/L 100 (DSM), ROHAMENT™ 7069 W (Röhm GmbH), FIBREZYME® LDI (Dyadic International, Inc.), FIBREZYME® LBR (Dyadic International, Inc.), or VISCOSTAR® 150L (Dyadic International, Inc.). The cellulase enzymes are added in amounts effective from about 0.001 to about 5.0 wt % of solids, e.g., about 0.025 to about 4.0 wt % of solids or about 0.005 to about 2.0 wt % of solids.

Examples of bacterial endoglucanases that can be used in the methods of the present invention, include, but are not limited to, an Acidothermus cellulolyticus endoglucanase (WO 91/05039; WO 93/15186; U.S. Pat. No. 5,275,944; WO 96/02551; U.S. Pat. No. 5,536,655, WO 00/70031, WO 05/093050); Thermobifida fusca endoglucanase III (WO 05/093050); and Thermobifida fusca endoglucanase V (WO 05/093050).

Examples of fungal endoglucanases that can be used in the present invention, include, but are not limited to, a Trichoderma reesei endoglucanase I (Penttila et al., 1986, Gene 45: 253-263, Trichoderma reesei Cel7B endoglucanase I (GENBANK™ accession no. M15665), Trichoderma reesei endoglucanase II (Saloheimo, et al., 1988, Gene 63:11-22), Trichoderma reesei CeI5A endoglucanase II (GENBANK™ accession no. M19373), Trichoderma reesei endoglucanase III (Okada et al., 1988, Appl. Environ. Microbiol. 64: 555-563, GENBANK™ accession no. AB003694), Trichoderma reesei endoglucanase V (Saloheimo et al., 1994, Molecular Microbiology 13: 219-228, GENBANK™ accession no. Z33381), Aspergillus aculeatus endoglucanase (Ooi et al., 1990, Nucleic Acids Research 18: 5884), Aspergillus kawachii endoglucanase (Sakamoto et al., 1995, Current Genetics 27: 435-439), Erwinia carotovara endoglucanase (Saarilahti et al., 1990, Gene 90: 9-14), Fusarium oxysporum endoglucanase (GENBANK™ accession no. L29381), Humicola grisea var. thermoidea endoglucanase (GENBANK™ accession no. AB003107), Melanocarpus albomyces endoglucanase (GENBANK™ accession no. MAL515703), Neurospora crassa endoglucanase (GENBANK™ accession no. XM_{—}324477), Humicola insolens endoglucanase V, Myceliophthora thermophila CBS 117.65 endoglucanase, basidiomycete CBS 495.95 endoglucanase, basidiomycete CBS 494.95 endoglucanase, Thielavia terrestris NRRL 8126 CEL6B endoglucanase, Thielavia terrestris NRRL 8126 CEL6C endoglucanase, Thielavia terrestris NRRL 8126 CEL7C endoglucanase, Thielavia terrestris NRRL 8126 CEL7E endoglucanase, Thielavia terrestris NRRL 8126 CEL7F endoglucanase, Cladorrhinum foecundissimum ATCC 62373 CEL7A endoglucanase, and Trichoderma reesei strain No. VTT-D-80133 endoglucanase (GENBANK™ accession no. M15665).

Examples of cellobiohydrolases useful in the present invention include, but are not limited to, Aspergillus aculeatus cellobiohydrolase II (WO 2011/059740), Chaetomium thermophilum cellobiohydrolase I, Chaetomium thermophilum cellobiohydrolase II, Humicola insolens cellobiohydrolase I, Myceliophthora thermophila cellobiohydrolase II (WO 2009/042871), Thielavia hyrcanie cellobiohydrolase II (WO 2010/141325), Thielavia terrestris cellobiohydrolase II (CEL6A, WO 2006/074435), Trichoderma reesei cellobiohydrolase I, Trichoderma reesei cellobiohydrolase II, and Trichophaea saccata cellobiohydrolase II (WO 2010/057086).

Examples of beta-glucosidases useful in the present invention include, but are not limited to, beta-glucosidases from Aspergillus aculeatus (Kawaguchi et al., 1996, Gene 173: 287-288), Aspergillus fumigatus (WO 2005/047499), Aspergillus niger (Dan et al., 2000, J. Biol. Chem. 275: 4973-4980), Aspergillus oryzae (WO 2002/095014), Penicillium brasilianum IBT 20888 (WO 2007/019442 and WO 2010/088387), Thielavia terrestris (WO 2011/035029), and Trichophaea saccata (WO 2007/019442).

The beta-glucosidase may be a fusion protein. In one aspect, the beta-glucosidase is an Aspergillus oryzae beta-glucosidase variant BG fusion protein (WO 2008/057637) or an Aspergillus oryzae beta-glucosidase fusion protein (WO 2008/057637).

Other useful endoglucanases, cellobiohydrolases, and beta-glucosidases are disclosed in numerous Glycosyl Hydrolase families using the classification according to Henrissat B., 1991, A classification of glycosyl hydrolases based on amino-acid sequence similarities, Biochem. J. 280: 309-316, and Henrissat B., and Bairoch A., 1996, Updating the sequence-based classification of glycosyl hydrolases, Biochem. J. 316: 695-696.

Other cellulolytic enzymes that may be used in the present invention are described in WO 98/13465, WO 98/015619, WO 98/015633, WO 99/06574, WO 99/10481, WO 99/025847, WO 99/031255, WO 2002/101078, WO 2003/027306, WO 2003/052054, WO 2003/052055, WO 2003/052056, WO 2003/052057, WO 2003/052118, WO 2004/016760, WO 2004/043980, WO 2004/048592, WO 2005/001065, WO 2005/028636, WO 2005/093050, WO 2005/093073, WO 2006/074005, WO 2006/117432, WO 2007/071818, WO 2007/071820, WO 2008/008070, WO 2008/008793, U.S. Pat. No. 5,457,046, U.S. Pat. No. 5,648,263, and U.S. Pat. No. 5,686,593.

In the methods of the present invention, any GH61 polypeptide having cellulolytic enhancing activity can be used as a component of the enzyme composition.

Examples of GH61 polypeptides having cellulolytic enhancing activity useful in the methods of the present invention include, but are not limited to, GH61 polypeptides from Thielavia terrestris (WO 2005/074647, WO 2008/148131, and WO 2011/035027), Thermoascus aurantiacus (WO 2005/074656 and WO 2010/065830), Trichoderma reesei (WO 2007/089290), Myceliophthora thermophila (WO 2009/085935, WO 2009/085859, WO 2009/085864, WO 2009/085868), Aspergillus fumigatus (WO 2010/138754), GH61 polypeptides from Penicillium pinophilum (WO 2011/005867), Thermoascus sp. (WO 2011/039319), Penicillium sp. (WO 2011/041397), and Thermoascus crustaceous (WO 2011/041504).

In one aspect, the GH61 polypeptide having cellulolytic enhancing activity is used in the presence of a soluble activating divalent metal cation according to WO 2008/151043, e.g., manganese sulfate.

In another aspect, the GH61 polypeptide having cellulolytic enhancing activity is used in the presence of a dioxy compound, a bicylic compound, a heterocyclic compound, a nitrogen-containing compound, a quinone compound, a sulfur-containing compound, or a liquor obtained from a pretreated cellulosic material such as pretreated corn stover (PCS).

The dioxy compound may include any suitable compound containing two or more oxygen atoms. In some aspects, the dioxy compounds contain a substituted aryl moiety as described herein. The dioxy compounds may comprise one or more (e.g., several) hydroxyl and/or hydroxyl derivatives, but also include substituted aryl moieties lacking hydroxyl and hydroxyl derivatives. Non-limiting examples of the dioxy compounds include pyrocatechol or catechol; caffeic acid; 3,4-dihydroxybenzoic acid; 4-tert-butyl-5-methoxy-1,2-benzenediol; pyrogallol; gallic acid; methyl-3,4,5-trihydroxybenzoate; 2,3,4-trihydroxybenzophenone; 2,6-dimethoxyphenol; sinapinic acid; 3,5-dihydroxybenzoic acid; 4-chloro-1,2-benzenediol; 4-nitro-1,2-benzenediol; tannic acid; ethyl gallate; methyl glycolate; dihydroxyfumaric acid; 2-butyne-1,4-diol; (croconic acid; 1,3-propanediol; tartaric acid; 2,4-pentanediol; 3-ethyoxy-1,2-propanediol; 2,4,4′-trihydroxybenzophenone; cis-2-butene-1,4-diol; 3,4-dihydroxy-3-cyclobutene-1,2-dione; dihydroxyacetone; acrolein acetal; methyl-4-hydroxybenzoate; 4-hydroxybenzoic acid; and methyl-3,5-dimethoxy-4-hydroxybenzoate; or a salt or solvate thereof.

The bicyclic compound may include any suitable substituted fused ring system as described herein. The compounds may comprise one or more (e.g., several) additional rings, and are not limited to a specific number of rings unless otherwise stated. In one aspect, the bicyclic compound is a flavonoid. In another aspect, the bicyclic compound is an optionally substituted isoflavonoid. In another aspect, the bicyclic compound is an optionally substituted flavylium ion, such as an optionally substituted anthocyanidin or optionally substituted anthocyanin, or derivative thereof. Non-limiting examples of the bicyclic compounds include epicatechin; quercetin; myricetin; taxifolin; kaempferol; morin; acacetin; naringenin; isorhamnetin; apigenin; cyanidin; cyanin; kuromanin; keracyanin; or a salt or solvate thereof.

The heterocyclic compound may be any suitable compound, such as an optionally substituted aromatic or non-aromatic ring comprising a heteroatom, as described herein. In one aspect, the heterocyclic is a compound comprising an optionally substituted heterocycloalkyl moiety or an optionally substituted heteroaryl moiety. In another aspect, the optionally substituted heterocycloalkyl moiety or optionally substituted heteroaryl moiety is an optionally substituted 5-membered heterocycloalkyl or an optionally substituted 5-membered heteroaryl moiety. In another aspect, the optionally substituted heterocycloalkyl or optionally substituted heteroaryl moiety is an optionally substituted moiety selected from pyrazolyl, furanyl, imidazolyl, isoxazolyl, oxadiazolyl, oxazolyl, pyrrolyl, pyridyl, pyrimidyl, pyridazinyl, thiazolyl, triazolyl, thienyl, dihydrothieno-pyrazolyl, thianaphthenyl, carbazolyl, benzimidazolyl, benzothienyl, benzofuranyl, indolyl, quinolinyl, benzotriazolyl, benzothiazolyl, benzooxazolyl, benzimidazolyl, isoquinolinyl, isoindolyl, acridinyl, benzoisazolyl, dimethylhydantoin, pyrazinyl, tetrahydrofuranyl, pyrrolinyl, pyrrolidinyl, morpholinyl, indolyl, diazepinyl, azepinyl, thiepinyl, piperidinyl, and oxepinyl. In another aspect, the optionally substituted heterocycloalkyl moiety or optionally substituted heteroaryl moiety is an optionally substituted furanyl. Non-limiting examples of the heterocyclic compounds include (1,2-dihydroxyethyl)-3,4-dihydroxyfuran-2(5H)-one; 4-hydroxy-5-methyl-3-furanone; 5-hydroxy-2(5H)-furanone; [1,2-dihydroxyethyl]furan-2,3,4(5H)-trione; α-hydroxy-γ-butyrolactone; ribonic γ-lactone; aldohexuronicaldohexuronic acid γ-lactone; gluconic acid δ-lactone; 4-hydroxycoumarin; dihydrobenzofuran; 5-(hydroxymethyl)furfural; furoin; 2(5H)-furanone; 5,6-dihydro-2H-pyran-2-one; and 5,6-dihydro-4-hydroxy-6-methyl-2H-pyran-2-one; or a salt or solvate thereof.

The nitrogen-containing compound may be any suitable compound with one or more nitrogen atoms. In one aspect, the nitrogen-containing compound comprises an amine, imine, hydroxylamine, or nitroxide moiety. Non-limiting examples of the nitrogen-containing compounds include acetone oxime; violuric acid; pyridine-2-aldoxime; 2-aminophenol; 1,2-benzenediamine; 2,2,6,6-tetramethyl-1-piperidinyloxy; 5,6,7,8-tetrahydrobiopterin; 6,7-dimethyl-5,6,7,8-tetrahydropterine; and maleamic acid; or a salt or solvate thereof.

The quinone compound may be any suitable compound comprising a quinone moiety as described herein. Non-limiting examples of the quinone compounds include 1,4-benzoquinone; 1,4-naphthoquinone; 2-hydroxy-1,4-naphthoquinone; 2,3-dimethoxy-5-methyl-1,4-benzoquinone or coenzyme Q₀; 2,3,5,6-tetramethyl-1,4-benzoquinone or duroquinone; 1,4-dihydroxyanthraquinone; 3-hydroxy-1-methyl-5,6-indolinedione or adrenochrome; 4-tert-butyl-5-methoxy-1,2-benzoquinone; pyrroloquinoline quinone; or a salt or solvate thereof.

The sulfur-containing compound may be any suitable compound comprising one or more sulfur atoms. In one aspect, the sulfur-containing comprises a moiety selected from thionyl, thioether, sulfinyl, sulfonyl, sulfamide, sulfonamide, sulfonic acid, and sulfonic ester. Non-limiting examples of the sulfur-containing compounds include ethanethiol; 2-propanethiol; 2-propene-1-thiol; 2-mercaptoethanesulfonic acid; benzenethiol; benzene-1,2-dithiol; cysteine; methionine; glutathione; cystine; or a salt or solvate thereof.

In one aspect, an effective amount of such a compound described above to cellulosic material as a molar ratio to glucosyl units of cellulose is about 10⁻⁶ to about 10, e.g., about 10⁻⁶ to about 7.5, about 10⁻⁶ to about 5, about 10⁻⁶ to about 2.5, about 10⁻⁶ to about 1, about 10⁻⁵ to about 1, about 10⁻⁵ to about 10⁻¹, about 10⁻⁴ to about 10⁻¹, about 10⁻³ to about 10⁻¹, or about 10⁻³ to about 10⁻². In another aspect, an effective amount of such a compound described above is about 0.1 μM to about 1 M, e.g., about 0.5 μM to about 0.75 M, about 0.75 μM to about 0.5 M, about 1 μM to about 0.25 M, about 1 μM to about 0.1 M, about 5 μM to about 50 mM, about 10 μM to about 25 mM, about 50 μM to about 25 mM, about 10 μM to about 10 mM, about 5 μM to about 5 mM, or about 0.1 mM to about 1 mM.

The term “liquor” means the solution phase, either aqueous, organic, or a combination thereof, arising from treatment of a lignocellulose and/or hemicellulose material in a slurry, or monosaccharides thereof, e.g., xylose, arabinose, mannose, etc., under conditions as described herein, and the soluble contents thereof. A liquor for cellulolytic enhancement of a GH61 polypeptide can be produced by treating a lignocellulose or hemicellulose material (or feedstock) by applying heat and/or pressure, optionally in the presence of a catalyst, e.g., acid, optionally in the presence of an organic solvent, and optionally in combination with physical disruption of the material, and then separating the solution from the residual solids. Such conditions determine the degree of cellulolytic enhancement obtainable through the combination of liquor and a GH61 polypeptide during hydrolysis of a cellulosic substrate by a cellulase preparation. The liquor can be separated from the treated material using a method standard in the art, such as filtration, sedimentation, or centrifugation.

In one aspect, an effective amount of the liquor to cellulose is about 10⁻⁶ to about 10 g per g of cellulose, e.g., about 10⁻⁶ to about 7.5 g, about 10⁻⁶ to about 5 g, about 10⁻⁶ to about 2.5 g, about 10⁻⁶ to about 1 g, about 10⁻⁵ to about 1 g, about 10⁻⁵ to about 10⁻¹ g, about 10⁻⁴ to about 10⁻¹ g, about 10⁻³ to about 10⁻¹ g, or about 10⁻³ to about 10⁻² g per g of cellulose.

In one aspect, the one or more (e.g., several) hemicellulolytic enzymes comprise a commercial hemicellulolytic enzyme preparation. Examples of commercial hemicellulolytic enzyme preparations suitable for use in the present invention include, for example, SHEARZYME™ (Novozymes NS), CELLIC® HTec (Novozymes NS), CELLIC® HTec2 (Novozymes NS), CELLIC® HTec3 (Novozymes NS), VISCOZYME® (Novozymes NS), ULTRAFLO® (Novozymes NS), PULPZYME® HC (Novozymes NS), MULTIFECT® Xylanase (Genencor), ACCELLERASE® XY (Genencor), ACCELLERASE® XC (Genencor), ECOPULP® TX-200A (AB Enzymes), HSP 6000 Xylanase (DSM), DEPOL™ 333P (Biocatalysts Limit, Wales, UK), DEPOL™ 740L. (Biocatalysts Limit, Wales, UK), and DEPOL™ 762P (Biocatalysts Limit, Wales, UK).

Examples of xylanases useful in the methods of the present invention include, but are not limited to, xylanases from Aspergillus aculeatus (GeneSeqP:AAR63790; WO 94/21785), Aspergillus fumigatus (WO 2006/078256), Penicillium pinophilum (WO 2011/041405), Penicillium sp. (WO 2010/126772), Thielavia terrestris NRRL 8126 (WO 2009/079210), and Trichophaea saccata GH10 (WO 2011/057083).

Examples of beta-xylosidases useful in the methods of the present invention include, but are not limited to, beta-xylosidases from Neurospora crassa(SwissProt accession number Q7SOW4), Trichoderma reesei (UniProtKB/TrEMBL accession number Q92458), and Talaromyces emersonii (SwissProt accession number Q8X212).

Examples of acetylxylan esterases useful in the methods of the present invention include, but are not limited to, acetylxylan esterases from Aspergillus aculeatus (WO 2010/108918), Chaetomium globosum (Uniprot accession number Q2GWX4), Chaetomium gracile (GeneSeqP accession number AAB82124), Humicola insolens DSM 1800 (WO 2009/073709), Hypocrea jecorina (WO 2005/001036), Myceliophtera thermophila (WO 2010/014880), Neurospora crassa(UniProt accession number q7s259), Phaeosphaeria nodorum (Uniprot accession number QOUHJ1), and Thielavia terrestris NRRL 8126 (WO 2009/042846).

Examples of feruloyl esterases (ferulic acid esterases) useful in the methods of the present invention include, but are not limited to, feruloyl esterases form Humicola insolens DSM 1800 (WO 2009/076122), Neosartorya fischeri (UniProt Accession number A1D9T4), Neurospora crassa(UniProt accession number Q9HGR3), Penicillium aurantiogriseum (WO 2009/127729), and Thielavia terrestris (WO 2010/053838 and WO 2010/065448).

Examples of arabinofuranosidases useful in the methods of the present invention include, but are not limited to, arabinofuranosidases from Aspergillus niger (GeneSeqP accession number AAR94170), Humicola insolens DSM 1800 (WO 2006/114094 and WO 2009/073383), and M. giganteus (WO 2006/114094).

Examples of alpha-glucuronidases useful in the methods of the present invention include, but are not limited to, alpha-glucuronidases from Aspergillus clavatus (UniProt accession number alcc12), Aspergillus fumigatus (SwissProt accession number Q4WW45), Aspergillus niger (Uniprot accession number Q96WX9), Aspergillus terreus (SwissProt accession number QOCJP9), Humicola insolens (WO 2010/014706), Penicillium aurantiogriseum (WO 2009/068565), Talaromyces emersonii (UniProt accession number Q8X211), and Trichoderma reesei (Uniprot accession number Q99024).

For starch materials, the enzyme composition can comprise any protein useful in degrading a starch material. In one aspect, the enzyme composition comprises or further comprises one or more (e.g., several) amylolytic enzymes. In another aspect, the enzyme composition comprises or further comprises one or more (e.g., several) amylolytic enzymes selected from the group consisting of an alpha-amylase, an amyloglucosidase, a maltogenic alpha-amylase, a beta-amylase, or a pullulanase. In another aspect, the enzyme composition comprises or further comprises an alpha-amylase. In another aspect, the enzyme composition comprises or further comprises an amyloglucosidase. In another aspect, the enzyme composition comprises or further comprises a maltogenic alpha-amylase. In another aspect, the enzyme composition comprises or further comprises a beta-amylase. In another aspect, the enzyme composition comprises or further comprises a pullulanase.

The alpha-amylase may be any alpha-amylase useful in the methods of the present invention. In one aspect, the alpha-amylase is an acid alpha-amylase, e.g., a fungal acid alpha-amylase or a bacterial acid alpha-amylase. The term “acid alpha-amylase” means an alpha-amylase (E.C. 3.2.1.1) having optimal activity at a pH in the range of preferably 3 to 7, more preferably 3.5 to 6, and most preferably 4 to 5.

A bacterial alpha-amylase is preferably derived from the genus Bacillus. In one aspect, the Bacillus alpha-amylase is derived from a strain of B. licheniformis, B. amyloliquefaciens, B. subtilis or B. stearothermophilus, but may also be derived from other Bacillus sp. Examples of alpha-amylases include Bacillus licheniformis alpha-amylase (WO 99/19467), Bacillus amyloliquefaciens alpha-amylase (WO 99/19467), and Bacillus stearothermophilus alpha-amylase (WO 99/19467).

A Bacillus alpha-amylase may also be a variant and/or hybrid, such as the variants and hybrids disclosed in WO 96/23873, WO 96/23874, WO 97/41213, WO 99/19467, WO 00/60059, and WO 02/10355. Examples of other alpha-amylase variants are disclosed in U.S. Pat. Nos. 6,093,562, 6,297,038, and 6,187,576.

Alpha-amylase activity may be determined using potato starch as substrate. This method is based on the break-down of modified potato starch by the enzyme, and the reaction is followed by mixing samples of the starch/enzyme solution with an iodine solution. Initially, a blackish-blue color is formed, but during the break-down of the starch the blue color gets weaker and gradually turns into a reddish-brown, which is compared to a colored standard. One Kilo Novo alpha amylase Unit (KNU) is defined as the amount of enzyme which, under standard conditions (i.e., at 37° C.+/−0.05; 0.0003 M Ca²⁺; and pH 5.6) dextrinizes 5260 mg starch dry substance Merck Amylum soluble.

A bacterial alpha-amylase is dosed at an amount of 0.0005-5 KNU per g DS (dry solids), preferably 0.001-1 KNU per g DS, such as around 0.050 KNU per g DS.

The alpha-amylase may also be a fungal alpha-amylase. Fungal alpha-amylases include alpha-amylases obtained from strains of the genus Aspergillus, such as, Aspergillus oryzae, Aspergillus niger and Aspergillus kawachii alpha-amylases.

A preferred acidic alpha-amylase is obtained from a strain Aspergillus niger. In an aspect, the acid fungal alpha-amylase is the alpha-amylase described in WO 89/01969 (Example 3). A commercially available acid fungal alpha-amylase derived from Aspergillus niger is SP288 (available from Novozymes NS, Denmark).

Other useful wild-type alpha-amylases include those obtained from strains of the genera Rhizomucor and Meripilus, preferably a strain of Rhizomucor pusillus (WO 2004/055178) or Meripilus giganteus.

In another aspect the alpha-amylase is derived from Aspergillus kawachii disclosed by Kaneko et al., 1996, J. Ferment. Bioeng. 81: 292-298, and further as EMBL #AB008370.

The fungal alpha-amylase may also be a wild-type enzyme comprising a starch-binding domain (SBD) and an alpha-amylase catalytic domain (i.e., non-hybrid), or a variant thereof. In one aspect the wild-type alpha-amylase is derived from a strain of Aspergillus kawachii.

The alpha-amylase may also be a fungal hybrid alpha-amylase. Preferred examples of fungal hybrid alpha-amylases include those disclosed in WO 2005/003311, U.S. Patent Application 2005/0054071, or U.S. Patent Application No. 2006/0148054. A hybrid alpha-amylase may comprise an alpha-amylase catalytic domain (CD) and a carbohydrate-binding domain/module (CBM), such as a starch binding domain, and optionally a linker.

Specific examples of hybrid alpha-amylases include those disclosed in Tables 1-5 of U.S. Patent Application No. 2006/0148054, including an Aspergillus oryzae alpha-amylase variant with a catalytic domain JA118 and an Athelia rolfsii SBD (U.S. Patent Application No. 2006/0148054), Rhizomucor pusillus alpha-amylase with an Athelia rolfsii AMG linker and a SBD (U.S. Patent Application No. 2006/0148054), Rhizomucor pusillus alpha-amylase with an Aspergillus niger glucoamylase linker and a SBD (which is disclosed in Table 5 of U.S. Patent Application No. 2006/0148054) or as V039 in Table 5 of WO 2006/069290, and a Meripilus giganteus alpha-amylase with an Athelia rolfsii glucoamylase linker and a SBD (U.S. Patent Application No. 2006/0148054). Other useful hybrid alpha-amylases are those listed in Tables 3, 4, 5, and 6 in U.S. Patent Application No. 2006/0148054 and WO 2006/069290.

Other specific examples of hybrid alpha-amylases include those disclosed in U.S. Patent Application 2005/0054071, including those disclosed in Table 3 thereof, such as Aspergillus niger alpha-amylase with an Aspergillus kawachii linker and a starch binding domain.

Preferred commercial compositions comprising alpha-amylase include MYCOLASE™ from DSM (Gist Brocades), BAN™, TERMAMYL™ SC, FUNGAMYL™, LIQUOZYME™ X, LIQUOZYME™ SC and SAN™ SUPER, SAN™ EXTRA L (Novozymes NS) and CLARASE™ L-40,000, DEX-LO™, SPEZYME® FRED-L, SPEZYME® HPA, SPEZYME® ALPHA, SPEZYME® XTRA, SPEZYME® AA, SPEZYME® DELTA AA, and GC358 (Genencor Int.), SPEZYME RSL, FUELZYME™-LF (Verenium Inc.), and the acid fungal alpha-amylase under the trade name SP288 (available from Novozymes NS, Denmark).

Acid alpha-amylase activity may be measured in AFAU (Acid Fungal Alpha-amylase Units), which are determined relative to an enzyme standard. One AFAU is defined as the amount of enzyme which degrades 5.260 mg starch dry matter per hour under standard conditions.

Fungal Alpha-Amylase Units (FAU-F) are measured relative to an enzyme standard of a declared strength.

In the methods of the present invention, an acid alpha-amylase may be added in an amount of preferably 0.1 to 10 AFAU/g DS, more preferably 0.10 to 5 AFAU/g DS, and most preferably 0.3 to 2 AFAU/g DS or preferably 0.001 to 1 FAU-F/g DS and more preferably 0.01 to 1 FAU-F/g DS.

The glucoamylase may be any glucoamylase useful in the methods of the present invention. The glucoamylase may be obtained from any suitable source, e.g., a microorganism or a plant. Preferred glucoamylases are of fungal or bacterial origin. Examples of fungal glucoamylase are Aspergillus glucoamylases, in particular, A. niger G1 or G2 glucoamylase (Boel et al., 1984, EMBO J. 3: 1097-1102), or variants thereof, such as those disclosed in WO 92/00381, WO 00/04136 and WO 01/04273; A. awamori glucoamylase disclosed in WO 84/02921, A. oryzae glucoamylase (Hata et al., 1991, Agric. Biol. Chem. 55: 941-949), or variants or fragments thereof. Other Aspergillus glucoamylase variants include variants with enhanced thermal stability (Chen et al., 1996, Prot. Eng. 9: 499-505; Chen et al., 1995, Prot. Eng. 8: 575-582; Chen et al., 1994, Biochem. J. 301: 275-281; Fierobe et al., 1996, Biochemistry 35: 8698-8704; and Li et al., 1997, Protein Eng. 10: 1199-1204).

Other useful glucoamylases include Athelia rolfsii (previously denoted Corticium rolfsii) glucoamylase (U.S. Pat. No. 4,727,026 and Nagasaka et al., 1998, Appl. Microbiol. Biotechnol. 50:323-330), Talaromyces glucoamylases, in particular, glucoamylases obtained from Talaromyces emersonii (WO 99/28448), Talaromyces leycettanus (U.S. Pat. No. Re. 32,153), Talaromyces duponti, and Talaromyces thermophilus (U.S. Pat. No. 4,587,215).

Other useful bacterial glucoamylases include glucoamylases from the genus Clostridium, in particular C. thermoamylolyticum (EP 135,138), and C. thermohydrosulfuricum (WO 86/01831), and Trametes cingulata disclosed in WO 2006/069289.

Also hybrid glucoamylases can be used in the methods of the present invention. Examples of hybrid glucoamylases are disclosed in WO 2005/045018.

Commercially available compositions comprising glucoamylase include AMG 200L; AMG 300 L; SAN™ SUPER, SAN™ EXTRA L, SPIRIZYME™ PLUS, SPIRIZYME™ FUEL, SPIRIZYME™ B4U, SPIRIZYME™ ULTRA, SPIRIZYME EXCEL, and AMG™ E (Novozymes NS); OPTIDEX™ 300, GC480, GC147 (Genencor Int.); AMIGASE™ and AMIGASE™ PLUS (DSM); G-ZYME® G900, G-ZYME®, G-ZYME® 480 ETHANOL, DISTILLASE® L-400, DISTILLASE® L-500, DISTILLASE® VHP, and G990 ZR (Genencor Int.).

The Novo Glucoamylase Unit (AGU) is defined as the amount of enzyme, which hydrolyzes 1 micromole of maltose per minute under standard conditions of 37° C., pH 4.3, using 23.2 mM maltose as substrate in 0.1 M acetate buffer for a reaction time of 5 minutes. An autoanalyzer system may be used where mutarotase is added to the glucose dehydrogenase reagent so that any alpha-D-glucose present is converted to beta-D-glucose. Glucose dehydrogenase reacts specifically with beta-D-glucose in the reaction mentioned above, forming NADH which is determined at 340 nm as a measure of the original glucose concentration.

Glucoamylases are preferably added in an amount of 0.0001-20 AGU/g DS, more preferably 0.001-10 AGU/g DS, 0.02-10 AGU/g DS, more preferably 0.1-10 AGU/g DS, even more preferably 0.1-5 AGU/g DS, and most preferably 0.1-2 AGU/g DS.

The beta-amylase may be any beta-amylase useful in the methods of the present invention. Beta-amylases have been isolated from various plants and microorganisms (W. M. Fogarty and C. T. Kelly, 1979, Progress in Industrial Microbiology 15: 112-115). These beta-amylases are characterized by having optimum temperatures in the range from 40° C. to 65° C. and optimum pH in the range from 4.5 to 7. Commercially available beta-amylases are NOVOZYM™ WBA from Novozymes NS, Denmark and SPEZYME™ BBA 1500 from Genencor Int., USA.

A beta-amylase Unit (BAMU) is defined as the amount of enzyme that degrades one pmole of maltohexose per minute, or defined as the activity presented by 1 mg of pure beta-amylase enzyme. One BAMU is defined relative to the BAMU of an enzyme standard.

The maltogenic alpha-amylase may be any maltogenic alpha-amylase useful in the methods of the present invention. Maltogenic alpha-amylases (glucan 1,4-alpha-maltohydrolase, E.C. 3.2.1.133) hydrolyze amylose and amylopectin to maltose in the alpha-configuration. A maltogenic amylase from Bacillus stearothermophilus strain NCIB 11837 is commercially available from Novozymes NS. Maltogenic alpha-amylases are described in U.S. Pat. Nos. 4,598,048, 4,604,355 and 6,162,628.

One MANU (Maltogenic Amylase Novo Unit) is defined as the amount of enzyme required to release one micromole of maltose per minute at a concentration of 10 mg of maltotriose (Sigma M 8378; Sigma Chemical Co., St. Louis, Mo., USA) substrate per ml of 0.1 M citrate pH 5.0 at 37° C. for 30 minutes.

The maltogenic amylase is preferably added in an amount of 0.05-5 mg total protein/gram DS or 0.05-5 MANU/g DS.

The pullulanase may be any pullulanase useful in the methods of the present invention. Pullulanases (E.C. 3.2.1.41), also known as pullulanan-6-glucanohydrolase, degrade alpha-1,6-linkages of pullulan, amylopectin, and other branched substrates. In the grain industry, bacterial pullulanases have been used for the purpose of removing alpha-1,6 bonds in starch, which may cause undesirable panose formation in the saccharification process.

A number of bacterial pullulanases are known. Kelly et al., 1994, FEMS Microbiology Letters 115: 97-106 describe the pullulanase B gene of Bacillus acidopullulyticus. WO 96/35794 describes a pullulanase from Bacillus sp.

Examples of commercial pullulanase products that can be used in the methods of the present invention include, but are not limited to, DEXTROZYME®, PROMOZYME® D2, and ATTENUZYME® from Novozymes NS.

Pullulanase activity may be determined relative to a pullulan substrate. Pullulan is a linear D-glucose polymer consisting essentially of maltotriosyl units joined by 1,6-alpha-links. Endo-pullulanases hydrolyze the 1,6-alpha-links at random, releasing maltotriose, 6³-alpha-maltotriosyl-maltotriose, 6³-alpha-(6³-alpha-maltotriosyl-maltotriosyl)-maltotriose. One New Pullulanase Unit Novo (NPUN) is a unit of endo-pullulanase activity and is measured relative to a Novozymes A/S PROMOZYME® standard. Standard conditions are 30 minutes reaction time at 40° C. and pH 4.5; and with 0.7% pullulan as substrate. The amount of red substrate degradation product is measured spectrophotometrically at 510 nm and is proportional to the endo-pullulanase activity in the sample. One NPUN equals the amount of enzyme which under the standard conditions liberates reducing carbohydrate with a reducing power equivalent to 2.86 micromole glucose per minute.

The pullulanase is preferably added in an amount of 0.05-5 NPUN/g DS.

The polypeptides having enzyme activity used in the methods of the present invention may be produced by fermentation of the above-noted microbial strains on a nutrient medium containing suitable carbon and nitrogen sources and inorganic salts, using procedures known in the art (see, e.g., Bennett, J. W. and LaSure, L. (eds.), More Gene Manipulations in Fungi, Academic Press, CA, 1991). Suitable media are available from commercial suppliers or may be prepared according to published compositions (e.g., in catalogues of the American Type Culture Collection). Temperature ranges and other conditions suitable for growth and enzyme production are known in the art (see, e.g., Bailey, J. E., and Ollis, D. F., Biochemical Engineering Fundamentals, McGraw-Hill Book Company, NY, 1986).

The fermentation can be any method of cultivation of a cell resulting in the expression or isolation of an enzyme or protein. Fermentation may, therefore, be understood as comprising shake flask cultivation, or small- or large-scale fermentation (including continuous, batch, fed-batch, or solid state fermentations) in laboratory or industrial fermentors performed in a suitable medium and under conditions allowing the enzyme to be expressed or isolated. The resulting enzymes produced by the methods described above may be recovered from the fermentation medium and purified by conventional procedures.

### Pretreatment

The biomass material may be subjected to any pretreatment known in the art before use in the methods of the present invention. The pretreatment methods described below are illustrative of cellulosic material, but can be used for any biomass material.

Any pretreatment process known in the art can be used to disrupt plant cell wall components of the cellulosic material (Chandra et al., 2007, Adv. Biochem. Engin./Biotechnol. 108: 67-93; Galbe and Zacchi, 2007, Pretreatment of lignocellulosic materials for efficient bioethanol production, Adv. Biochem. Engin./Biotechnol. 108: 41-65; Hendriks and Zeeman, 2009, Pretreatments to enhance the digestibility of lignocellulosic biomass, Bioresource Technol. 100: 10-18; Mosier et al., 2005, Features of promising technologies for pretreatment of lignocellulosic biomass, Bioresource Technol. 96: 673-686; Taherzadeh and Karimi, 2008, Pretreatment of lignocellulosic wastes to improve ethanol and biogas production: A review, Int. J. of Mol. Sci. 9: 1621-1651; Yang and Wyman, 2008, Pretreatment: the key to unlocking low-cost cellulosic ethanol, Biofuels Bioproducts and Biorefining-Biofpr. 2: 26-40).

The cellulosic material can also be subjected to particle size reduction, sieving, pre-soaking, wetting, washing, and/or conditioning prior to pretreatment using methods known in the art.

Conventional pretreatments include, but are not limited to, steam pretreatment (with or without explosion), dilute acid pretreatment, hot water pretreatment, alkaline pretreatment, lime pretreatment, wet oxidation, wet explosion, ammonia fiber explosion, organosolv pretreatment, and biological pretreatment. Additional pretreatments include ammonia percolation, ultrasound, electroporation, microwave, supercritical CO₂, supercritical H₂O, ozone, ionic liquid, and gamma irradiation pretreatments.

Steam Pretreatment. In steam pretreatment, the cellulosic material is heated to disrupt the plant cell wall components, including lignin, hemicellulose, and cellulose to make the cellulose and other fractions, e.g., hemicellulose, accessible to enzymes. The cellulosic material is passed to or through a reaction vessel where steam is injected to increase the temperature to the required temperature and pressure and is retained therein for the desired reaction time. Steam pretreatment is preferably performed at 140-250° C., e.g., 160-200° C. or 170-190° C., where the optimal temperature range depends on addition of a chemical catalyst. Residence time for the steam pretreatment is preferably 1-60 minutes, e.g., 1-30 minutes, 1-20 minutes, 3-12 minutes, or 4-10 minutes, where the optimal residence time depends on temperature range and addition of a chemical catalyst. Steam pretreatment allows for relatively high solids loadings, so that the cellulosic material is generally only moist during the pretreatment. The steam pretreatment is often combined with an explosive discharge of the material after the pretreatment, which is known as steam explosion, that is, rapid flashing to atmospheric pressure and turbulent flow of the material to increase the accessible surface area by fragmentation (Duff and Murray, 1996, Bioresource Technology 855: 1-33; Galbe and Zacchi, 2002, Appl. Microbiol. Biotechnol. 59: 618-628; U.S. Patent Application No. 20020164730). During steam pretreatment, hemicellulose acetyl groups are cleaved and the resulting acid autocatalyzes partial hydrolysis of the hemicellulose to monosaccharides and oligosaccharides. Lignin is removed to only a limited extent.

Chemical Pretreatment: The term “chemical treatment” refers to any chemical pretreatment that promotes the separation and/or release of cellulose, hemicellulose, and/or lignin. Such a pretreatment can convert crystalline cellulose to amorphous cellulose. Examples of suitable chemical pretreatment processes include, for example, dilute acid pretreatment, lime pretreatment, wet oxidation, ammonia fiber/freeze explosion (AFEX), ammonia percolation (APR), ionic liquid, and organosolv pretreatments.

A catalyst such as H₂SO₄ or SO₂ (typically 0.3 to 5% w/w) is often added prior to steam pretreatment, which decreases the time and temperature, increases the recovery, and improves enzymatic hydrolysis (Ballesteros et al., 2006, Appl. Biochem. Biotechnol. 129-132: 496-508; Varga et al., 2004, Appl. Biochem. Biotechnol. 113-116: 509-523; Sassner et al., 2006, Enzyme Microb. Technol. 39: 756-762). In dilute acid pretreatment, the cellulosic material is mixed with dilute acid, typically H₂SO₄, and water to form a slurry, heated by steam to the desired temperature, and after a residence time flashed to atmospheric pressure. The dilute acid pretreatment can be performed with a number of reactor designs, e.g., plug-flow reactors, counter-current reactors, or continuous counter-current shrinking bed reactors (Duff and Murray, 1996, supra; Schell et al., 2004, Bioresource Technol. 91: 179-188; Lee et al., 1999, Adv. Biochem. Eng. Biotechnol. 65: 93-115).

Several methods of pretreatment under alkaline conditions can also be used. These alkaline pretreatments include, but are not limited to, sodium hydroxide, lime, wet oxidation, ammonia percolation (APR), and ammonia fiber/freeze explosion (AFEX).

Lime pretreatment is performed with calcium oxide or calcium hydroxide at temperatures of 85-150° C. and residence times from 1 hour to several days (Wyman et al., 2005, Bioresource Technol. 96: 1959-1966; Mosier et al., 2005, Bioresource Technol. 96: 673-686). WO 2006/110891, WO 2006/110899, WO 2006/110900, and WO 2006/110901 disclose pretreatment methods using ammonia.

Wet oxidation is a thermal pretreatment performed typically at 180-200° C. for 5-15 minutes with addition of an oxidative agent such as hydrogen peroxide or over-pressure of oxygen (Schmidt and Thomsen, 1998, Bioresource Technol. 64: 139-151; Palonen et al., 2004, Appl. Biochem. Biotechnol. 117: 1-17; Varga et al., 2004, Biotechnol. Bioeng. 88: 567-574; Martin et al., 2006, J. Chem. Technol. Biotechnol. 81: 1669-1677). The pretreatment is performed preferably at 1-40% dry matter, e.g., 2-30% dry matter or 5-20% dry matter, and often the initial pH is increased by the addition of alkali such as sodium carbonate.

A modification of the wet oxidation pretreatment method, known as wet explosion (combination of wet oxidation and steam explosion) can handle dry matter up to 30%. In wet explosion, the oxidizing agent is introduced during pretreatment after a certain residence time. The pretreatment is then ended by flashing to atmospheric pressure (WO 2006/032282).

Ammonia fiber explosion (AFEX) involves treating the cellulosic material with liquid or gaseous ammonia at moderate temperatures such as 90-150° C. and high pressure such as 17-20 bar for 5-10 minutes, where the dry matter content can be as high as 60% (Gollapalli et al., 2002, Appl. Biochem. Biotechnol. 98: 23-35; Chundawat et al., 2007, Biotechnol. Bioeng. 96: 219-231; Alizadeh et al., 2005, Appl. Biochem. Biotechnol. 121: 1133-1141; Teymouri et al., 2005, Bioresource Technol. 96: 2014-2018). During AFEX pretreatment cellulose and hemicelluloses remain relatively intact. Lignin-carbohydrate complexes are cleaved.

Organosolv pretreatment delignifies the cellulosic material by extraction using aqueous ethanol (40-60% ethanol) at 160-200° C. for 30-60 minutes (Pan et al., 2005, Biotechnol. Bioeng. 90: 473-481; Pan et al., 2006, Biotechnol. Bioeng. 94: 851-861; Kurabi et al., 2005, Appl. Biochem. Biotechnol. 121: 219-230). Sulphuric acid is usually added as a catalyst. In organosolv pretreatment, the majority of hemicellulose and lignin is removed.

Other examples of suitable pretreatment methods are described by Schell et al., 2003, Appl. Biochem. and Biotechnol. Vol. 105-108, p. 69-85, and Mosier et al., 2005, Bioresource Technology 96: 673-686, and U.S. Published Application 2002/0164730.

In one aspect, the chemical pretreatment is preferably carried out as a dilute acid treatment, and more preferably as a continuous dilute acid treatment. The acid is typically sulfuric acid, but other acids can also be used, such as acetic acid, citric acid, nitric acid, phosphoric acid, tartaric acid, succinic acid, hydrogen chloride, or mixtures thereof. Mild acid treatment is conducted in the pH range of preferably 1-5, e.g., 1-4 or 1-2.5. In one aspect, the acid concentration is in the range from preferably 0.01 to 10 wt % acid, e.g., 0.05 to 5 wt % acid or 0.1 to 2 wt % acid. The acid is contacted with the cellulosic material and held at a temperature in the range of preferably 140-200° C., e.g., 165-190° C., for periods ranging from 1 to 60 minutes.

In another aspect, pretreatment takes place in a slurry. In preferred aspects, the cellulosic material is present during pretreatment in amounts preferably between 10-80 wt %, e.g., 20-70 wt % or 30-60 wt %, such as around 40 wt %. The pretreated cellulosic material can be unwashed or washed using any method known in the art, e.g., washed with water.

Mechanical Pretreatment or Physical Pretreatment: The term “mechanical pretreatment” or “physical pretreatment” refers to any pretreatment that promotes size reduction of particles. For example, such pretreatment can involve various types of grinding or milling (e.g., dry milling, wet milling, or vibratory ball milling).

The cellulosic material can be pretreated both physically (mechanically) and chemically. Mechanical or physical pretreatment can be coupled with steaming/steam explosion, hydrothermolysis, dilute or mild acid treatment, high temperature, high pressure treatment, irradiation (e.g., microwave irradiation), or combinations thereof. In one aspect, high pressure means pressure in the range of preferably about 100 to about 400 psi, e.g., about 150 to about 250 psi. In another aspect, high temperature means temperatures in the range of about 100 to about 300° C., e.g., about 140 to about 200° C. In a preferred aspect, mechanical or physical pretreatment is performed in a batch-process using a steam gun hydrolyzer system that uses high pressure and high temperature as defined above, e.g., a Sunds Hydrolyzer available from Sunds Defibrator AB, Sweden. The physical and chemical pretreatments can be carried out sequentially or simultaneously, as desired.

Accordingly, in a preferred aspect, the cellulosic material is subjected to physical (mechanical) or chemical pretreatment, or any combination thereof, to promote the separation and/or release of cellulose, hemicellulose, and/or lignin.

Biological Pretreatment: The term “biological pretreatment” refers to any biological pretreatment that promotes the separation and/or release of cellulose, hemicellulose, and/or lignin from the cellulosic material. Biological pretreatment techniques can involve applying lignin-solubilizing microorganisms and/or enzymes (see, for example, Hsu, T.-A., 1996, Pretreatment of biomass, in Handbook on Bioethanol: Production and Utilization, Wyman, C. E., ed., Taylor & Francis, Washington, DC, 179-212; Ghosh and Singh, 1993, Physicochemical and biological treatments for enzymatic/microbial conversion of cellulosic biomass, Adv. Appl. Microbiol. 39: 295-333; McMillan, J. D., 1994, Pretreating lignocellulosic biomass: a review, in Enzymatic Conversion of Biomass for Fuels Production, Himmel, M. E., Baker, J. 0., and Overend, R. P., eds., ACS Symposium Series 566, American Chemical Society, Washington, DC, chapter 15; Gong, C. S., Cao, N. J., Du, J., and Tsao, G. T., 1999, Ethanol production from renewable resources, in Advances in Biochemical Engineering/Biotechnology, Scheper, T., ed., Springer-Verlag Berlin Heidelberg, Germany, 65: 207-241; Olsson and Hahn-Hagerdal, 1996, Fermentation of lignocellulosic hydrolysates for ethanol production, Enz. Microb. Tech. 18: 312-331; and Vallander and Eriksson, 1990, Production of ethanol from lignocellulosic materials: State of the art, Adv. Biochem. Eng./Biotechnol. 42: 63-95).

### Fermentation

The hydrolyzed or degraded biomass material may be subjected to fermentation. The fermentation can be performed in a multi-well reaction plate or the contents of the hydrolyzed or degraded biomass material in the wells of the multi-well reaction plate can be transferred to different vessels. The fermentation methods described below are illustrative of cellulosic material, but can be used for any biomass material.

The fermentable sugars obtained from the hydrolyzed biomass material, e.g., cellulosic material, can be fermented by one or more (e.g., several) fermenting microorganisms capable of fermenting the sugars directly or indirectly into a desired fermentation product. “Fermentation” or “fermentation process” refers to any fermentation process or any process comprising a fermentation step. Fermentation processes also include fermentation processes used in the consumable alcohol industry (e.g., beer and wine), dairy industry (e.g., fermented dairy products), leather industry, and tobacco industry. The fermentation conditions depend on the desired fermentation product and fermenting organism and can easily be determined by one skilled in the art.

In the fermentation, sugars, released from the material as a result of the pretreatment and enzymatic hydrolysis steps, are fermented to a product, e.g., ethanol, by a fermenting organism, such as yeast. Hydrolysis (saccharification) and fermentation can be separate or simultaneous, as described herein.

Any suitable hydrolyzed material can be used in the fermentation in practicing the present invention. The material is generally selected based on the desired fermentation product, i.e., the substance to be obtained from the fermentation, and the process employed, as is well known in the art.

The term “fermentation medium” is understood herein to refer to a medium before the fermenting microorganism(s) is(are) added, such as, a medium resulting from a saccharification process, as well as a medium used in a simultaneous saccharification and fermentation process (SSF).

“Fermenting microorganism” refers to any microorganism, including bacterial and fungal organisms, suitable for use in a desired fermentation process to produce a fermentation product. The fermenting organism can be hexose and/or pentose fermenting organisms, or a combination thereof. Both hexose and pentose fermenting organisms are well known in the art. Suitable fermenting microorganisms are able to ferment, i.e., convert, sugars, such as glucose, xylose, xylulose, arabinose, maltose, mannose, galactose, and/or oligosaccharides, directly or indirectly into the desired fermentation product. Examples of bacterial and fungal fermenting organisms producing ethanol are described by Lin et al., 2006, Appl. Microbiol. Biotechnol. 69: 627-642.

Examples of fermenting microorganisms that can ferment hexose sugars include bacterial and fungal organisms, such as yeast. Preferred yeast includes strains of Candida, Kluyveromyces, and Saccharomyces, e.g., Candida sonorensis, Kluyveromyces marxianus, and Saccharomyces cerevisiae.

Examples of fermenting organisms that can ferment pentose sugars in their native state include bacterial and fungal organisms, such as some yeast. Preferred xylose fermenting yeast include strains of Candida, preferably C. sheatae or C. sonorensis; and strains of Pichia, preferably P. stipitis, such as P. stipitis CBS 5773. Preferred pentose fermenting yeast include strains of Pachysolen, preferably P. tannophilus. Organisms not capable of fermenting pentose sugars, such as xylose and arabinose, may be genetically modified to do so by methods known in the art.

Examples of bacteria that can efficiently ferment hexose and pentose to ethanol include, for example, Bacillus coagulans, Clostridium acetobutylicum, Clostridium thermocellum, Clostridium phytofermentans, Geobacillus sp., Thermoanaerobacter saccharolyticum, and Zymomonas mobilis (Philippidis, 1996, supra).

Other fermenting organisms include strains of Bacillus, such as Bacillus coagulans; Candida, such as C. sonorensis, C. methanosorbosa, C. diddensiae, C. parapsilosis, C. naedodendra, C. blankii, C. entomophilia, C. brassicae, C. pseudotropicalis, C. boidinii, C. utilis, and C. scehatae; Clostridium, such as C. acetobutylicum, C. thermocellum, and C. phytofermentans; E. coli, especially E. coli strains that have been genetically modified to improve the yield of ethanol; Geobacillus sp.; Hansenula, such as Hansenula anomala; Klebsiella, such as K. oxytoca; Kluyveromyces, such as K. marxianus, K lactis, K. thermotolerans, and K. fragilis; Schizosaccharomyces, such as S. pombe; Thermoanaerobacter, such as Thermoanaerobacter saccharolyticum; and Zymomonas, such as Zymomonas mobilis.

In a preferred aspect, the yeast is a Bretannomyces. In a more preferred aspect, the yeast is Bretannomyces clausenii. In another preferred aspect, the yeast is a Candida. In another more preferred aspect, the yeast is Candida sonorensis. In another more preferred aspect, the yeast is Candida boidinii. In another more preferred aspect, the yeast is Candida blankii. In another more preferred aspect, the yeast is Candida brassicae. In another more preferred aspect, the yeast is Candida diddensii. In another more preferred aspect, the yeast is Candida entomophiliia. In another more preferred aspect, the yeast is Candida pseudotropicalis. In another more preferred aspect, the yeast is Candida scehatae. In another more preferred aspect, the yeast is Candida utilis. In another preferred aspect, the yeast is a Clavispora. In another more preferred aspect, the yeast is Clavispora lusitaniae. In another more preferred aspect, the yeast is Clavispora opuntiae. In another preferred aspect, the yeast is a Kluyveromyces. In another more preferred aspect, the yeast is Kluyveromyces fragilis. In another more preferred aspect, the yeast is Kluyveromyces marxianus. In another more preferred aspect, the yeast is Kluyveromyces thermotolerans. In another preferred aspect, the yeast is a Pachysolen. In another more preferred aspect, the yeast is Pachysolen tannophilus. In another preferred aspect, the yeast is a Pichia. In another more preferred aspect, the yeast is a Pichia stipitis. In another preferred aspect, the yeast is a Saccharomyces spp. In another more preferred aspect, the yeast is Saccharomyces cerevisiae. In another more preferred aspect, the yeast is Saccharomyces distaticus. In another more preferred aspect, the yeast is Saccharomyces uvarum.

In a preferred aspect, the bacterium is a Bacillus. In a more preferred aspect, the bacterium is Bacillus coagulans. In another preferred aspect, the bacterium is a Clostridium. In another more preferred aspect, the bacterium is Clostridium acetobutylicum. In another more preferred aspect, the bacterium is Clostridium phytofermentans. In another more preferred aspect, the bacterium is Clostridium thermocellum. In another more preferred aspect, the bacterium is Geobacillus sp. In another more preferred aspect, the bacterium is a Thermoanaerobacter. In another more preferred aspect, the bacterium is Thermoanaerobacter saccharolyticum. In another preferred aspect, the bacterium is a Zymomonas. In another more preferred aspect, the bacterium is Zymomonas mobilis.

Commercially available yeast suitable for ethanol production include, e.g., BIOFERM™ AFT and XR (NABC—North American Bioproducts Corporation, GA, USA), ETHANOL RED™ yeast (Fermentis/Lesaffre, USA), FALI™ (Fleischmann's Yeast, USA), FERMIOL™ (DSM Specialties), GERT STRAND™ (Gert Strand AB, Sweden), and SUPERSTART™ and THERMOSACC™ fresh yeast (Ethanol Technology, Wis., USA).

In a preferred aspect, the fermenting microorganism has been genetically modified to provide the ability to ferment pentose sugars, such as xylose utilizing, arabinose utilizing, and xylose and arabinose co-utilizing microorganisms.

The cloning of heterologous genes into various fermenting microorganisms has led to the construction of organisms capable of converting hexoses and pentoses to ethanol (co-fermentation) (Chen and Ho, 1993, Cloning and improving the expression of Pichia stipitis xylose reductase gene in Saccharomyces cerevisiae, Appl. Biochem. Biotechnol. 39-40: 135-147; Ho et al., 1998, Genetically engineered Saccharomyces yeast capable of effectively cofermenting glucose and xylose, Appl. Environ. Microbiol. 64: 1852-1859; Kotter and Ciriacy, 1993, Xylose fermentation by Saccharomyces cerevisiae, Appl. Microbiol. Biotechnol. 38: 776-783; Walfridsson et al., 1995, Xylose-metabolizing Saccharomyces cerevisiae strains overexpressing the TKL1 and TAL1 genes encoding the pentose phosphate pathway enzymes transketolase and transaldolase, Appl. Environ. Microbiol. 61: 4184-4190; Kuyper et al., 2004, Minimal metabolic engineering of Saccharomyces cerevisiae for efficient anaerobic xylose fermentation: a proof of principle, FEMS Yeast Research 4: 655-664; Beall et al., 1991, Parametric studies of ethanol production from xylose and other sugars by recombinant Escherichia coli, Biotech. Bioeng. 38: 296-303; Ingram et al., 1998, Metabolic engineering of bacteria for ethanol production, Biotechnol. Bioeng. 58: 204-214; Zhang et al., 1995, Metabolic engineering of a pentose metabolism pathway in ethanologenic Zymomonas mobilis, Science 267: 240-243; Deanda et al., 1996, Development of an arabinose-fermenting Zymomonas mobilis strain by metabolic pathway engineering, Appl. Environ. Microbiol. 62: 4465-4470; WO 2003/062430, xylose isomerase).

In a preferred aspect, the genetically modified fermenting microorganism is Candida sonorensis. In another preferred aspect, the genetically modified fermenting microorganism is Escherichia coli. In another preferred aspect, the genetically modified fermenting microorganism is Klebsiella oxytoca. In another preferred aspect, the genetically modified fermenting microorganism is Kluyveromyces marxianus. In another preferred aspect, the genetically modified fermenting microorganism is Saccharomyces cerevisiae. In another preferred aspect, the genetically modified fermenting microorganism is Zymomonas mobilis.

It is well known in the art that the organisms described above can also be used to produce other substances, as described herein.

The fermenting microorganism is typically added to the degraded cellulosic material or hydrolysate and the fermentation is performed for about 8 to about 96 hours, e.g., about 24 to about 60 hours. The temperature is typically between about 26° C. to about 60° C., e.g., about 32° C. or 50° C., and about pH 3 to about pH 8, e.g., pH 4-5, 6, or 7.

In one aspect, the yeast and/or another microorganism are applied to the degraded cellulosic material and the fermentation is performed for about 12 to about 96 hours, such as typically 24-60 hours. In another aspect, the temperature is preferably between about 20° C. to about 60° C., e.g., about 25° C. to about 50° C., about 32° C. to about 50° C., or about 32° C. to about 50° C., and the pH is generally from about pH 3 to about pH 7, e.g., about pH 4 to about pH 7. However, some fermenting organisms, e.g., bacteria, have higher fermentation temperature optima. Yeast or another microorganism is preferably applied in amounts of approximately 10⁵ to 10¹², preferably from approximately 10⁷ to 10¹⁰, especially approximately 2×10⁸ viable cell count per ml of fermentation broth. Further guidance in respect of using yeast for fermentation can be found in, e.g., “The Alcohol Textbook” (Editors K. Jacques, T. P. Lyons and D. R. Kelsall, Nottingham University Press, United Kingdom 1999), which is hereby incorporated by reference.

A fermentation stimulator can be used in combination with any of the processes described herein to further improve the fermentation process, and in particular, the performance of the fermenting microorganism, such as, rate enhancement and ethanol yield.

A “fermentation stimulator” refers to stimulators for growth of the fermenting microorganisms, in particular, yeast. Preferred fermentation stimulators for growth include vitamins and minerals. Examples of vitamins include multivitamins, biotin, pantothenate, nicotinic acid, meso-inositol, thiamine, pyridoxine, para-aminobenzoic acid, folic acid, riboflavin, and Vitamins A, B, C, D, and E. See, for example, Alfenore et al., Improving ethanol production and viability of Saccharomyces cerevisiae by a vitamin feeding strategy during fed-batch process, Springer-Verlag (2002), which is hereby incorporated by reference. Examples of minerals include minerals and mineral salts that can supply nutrients comprising P, K, Mg, S, Ca, Fe, Zn, Mn, and Cu.

A fermentation product can be any substance derived from the fermentation. The fermentation product can be, without limitation, an alcohol (e.g., arabinitol, n-butanol, isobutanol, ethanol, glycerol, methanol, ethylene glycol, 1,3-propanediol [propylene glycol], butanediol, glycerin, sorbitol, and xylitol); an alkane (e.g., pentane, hexane, heptane, octane, nonane, decane, undecane, and dodecane), a cycloalkane (e.g., cyclopentane, cyclohexane, cycloheptane, and cyclooctane), an alkene (e.g. pentene, hexene, heptene, and octene); an amino acid (e.g., aspartic acid, glutamic acid, glycine, lysine, serine, and threonine); a gas (e.g., methane, hydrogen (H₂), carbon dioxide (CO₂), and carbon monoxide (CO)); isoprene; a ketone (e.g., acetone); an organic acid (e.g., acetic acid, acetonic acid, adipic acid, ascorbic acid, citric acid, 2,5-diketo-D-gluconic acid, formic acid, fumaric acid, glucaric acid, gluconic acid, glucuronic acid, glutaric acid, 3-hydroxypropionic acid, itaconic acid, lactic acid, malic acid, malonic acid, oxalic acid, oxaloacetic acid, propionic acid, succinic acid, and xylonic acid); and polyketide. The fermentation product can also be protein as a high value product.

In a preferred aspect, the fermentation product is an alcohol. It will be understood that the term “alcohol” encompasses a substance that contains one or more hydroxyl moieties. In a more preferred aspect, the alcohol is n-butanol. In another more preferred aspect, the alcohol is isobutanol. In another more preferred aspect, the alcohol is ethanol. In another more preferred aspect, the alcohol is methanol. In another more preferred aspect, the alcohol is arabinitol. In another more preferred aspect, the alcohol is butanediol. In another more preferred aspect, the alcohol is ethylene glycol. In another more preferred aspect, the alcohol is glycerin. In another more preferred aspect, the alcohol is glycerol. In another more preferred aspect, the alcohol is 1,3-propanediol. In another more preferred aspect, the alcohol is sorbitol. In another more preferred aspect, the alcohol is xylitol. See, for example, Gong, C. S., Cao, N. J., Du, J., and Tsao, G. T., 1999, Ethanol production from renewable resources, in Advances in Biochemical Engineering/Biotechnology, Scheper, T., ed., Springer-Verlag Berlin Heidelberg, Germany, 65: 207-241; Silveira, M. M., and Jonas, R., 2002, The biotechnological production of sorbitol, Appl. Microbiol. Biotechnol. 59: 400-408; Nigam, P., and Singh, D., 1995, Processes for fermentative production of xylitol—a sugar substitute, Process Biochemistry 30 (2): 117-124; Ezeji, T. C., Qureshi, N. and Blaschek, H. P., 2003, Production of acetone, butanol and ethanol by Clostridium beijerinckii BA101 and in situ recovery by gas stripping, World Journal of Microbiology and Biotechnology 19 (6): 595-603.

In another preferred aspect, the fermentation product is an alkane. The alkane can be an unbranched or a branched alkane. In another more preferred aspect, the alkane is pentane. In another more preferred aspect, the alkane is hexane. In another more preferred aspect, the alkane is heptane. In another more preferred aspect, the alkane is octane. In another more preferred aspect, the alkane is nonane. In another more preferred aspect, the alkane is decane. In another more preferred aspect, the alkane is undecane. In another more preferred aspect, the alkane is dodecane.

In another preferred aspect, the fermentation product is a cycloalkane. In another more preferred aspect, the cycloalkane is cyclopentane. In another more preferred aspect, the cycloalkane is cyclohexane. In another more preferred aspect, the cycloalkane is cycloheptane. In another more preferred aspect, the cycloalkane is cyclooctane.

In another preferred aspect, the fermentation product is an alkene. The alkene can be an unbranched or a branched alkene. In another more preferred aspect, the alkene is pentene. In another more preferred aspect, the alkene is hexene. In another more preferred aspect, the alkene is heptene. In another more preferred aspect, the alkene is octene.

In another preferred aspect, the fermentation product is an amino acid. In another more preferred aspect, the organic acid is aspartic acid. In another more preferred aspect, the amino acid is glutamic acid. In another more preferred aspect, the amino acid is glycine. In another more preferred aspect, the amino acid is lysine. In another more preferred aspect, the amino acid is serine. In another more preferred aspect, the amino acid is threonine. See, for example, Richard, A., and Margaritis, A., 2004, Empirical modeling of batch fermentation kinetics for poly(glutamic acid) production and other microbial biopolymers, Biotechnology and Bioengineering 87 (4): 501-515.

In another preferred aspect, the fermentation product is a gas. In another more preferred aspect, the gas is methane. In another more preferred aspect, the gas is H₂. In another more preferred aspect, the gas is CO₂. In another more preferred aspect, the gas is CO. See, for example, Kataoka, N., A. Miya, and K. Kiriyama, 1997, Studies on hydrogen production by continuous culture system of hydrogen-producing anaerobic bacteria, Water Science and Technology 36 (6-7): 41-47; and Gunaseelan V. N. in Biomass and Bioenergy, Vol. 13 (1-2), pp. 83-114, 1997, Anaerobic digestion of biomass for methane production: A review.

In another preferred aspect, the fermentation product is isoprene.

In another preferred aspect, the fermentation product is a ketone. It will be understood that the term “ketone” encompasses a substance that contains one or more ketone moieties. In another more preferred aspect, the ketone is acetone. See, for example, Qureshi and Blaschek, 2003, supra.

In another preferred aspect, the fermentation product is an organic acid. In another more preferred aspect, the organic acid is acetic acid. In another more preferred aspect, the organic acid is acetonic acid. In another more preferred aspect, the organic acid is adipic acid. In another more preferred aspect, the organic acid is ascorbic acid. In another more preferred aspect, the organic acid is citric acid. In another more preferred aspect, the organic acid is 2,5-diketo-D-gluconic acid. In another more preferred aspect, the organic acid is formic acid. In another more preferred aspect, the organic acid is fumaric acid. In another more preferred aspect, the organic acid is glucaric acid. In another more preferred aspect, the organic acid is gluconic acid. In another more preferred aspect, the organic acid is glucuronic acid. In another more preferred aspect, the organic acid is glutaric acid. In another preferred aspect, the organic acid is 3-hydroxypropionic acid. In another more preferred aspect, the organic acid is itaconic acid. In another more preferred aspect, the organic acid is lactic acid. In another more preferred aspect, the organic acid is malic acid. In another more preferred aspect, the organic acid is malonic acid. In another more preferred aspect, the organic acid is oxalic acid. In another more preferred aspect, the organic acid is propionic acid. In another more preferred aspect, the organic acid is succinic acid. In another more preferred aspect, the organic acid is xylonic acid. See, for example, Chen, R., and Lee, Y. Y., 1997, Membrane-mediated extractive fermentation for lactic acid production from cellulosic biomass, Appl. Biochem. Biotechnol. 63-65: 435-448.

In another preferred aspect, the fermentation product is polyketide.

The present invention is further described by the following examples that should not be construed as limiting the scope of the invention.

### EXAMPLES

### Example 1

### Pretreatment of Corn Stover

Corn stover was pretreated at the U.S. Department of Energy National Renewable Energy Laboratory (NREL) using 1.4% (w/v) sulfuric acid for 8 minutes at 165° C. and 107 psi. The water-insoluble solids in the pretreated corn stover contained 57.5% cellulose, 4.6% hemicelluloses, and 28.4% lignin. Cellulose and hemicellulose were determined by a two-stage sulfuric acid hydrolysis with subsequent analysis of sugars by high performance liquid chromatography using NREL Standard Analytical Procedure #002. Lignin was determined gravimetrically after hydrolyzing the cellulose and hemicellulose fractions with sulfuric acid using NREL Standard Analytical Procedure #003.

The pretreated corn stover was adjusted to pH 5.0 by repeated addition of 10 N NaOH in aliquots of a few milliliters, followed by thorough mixing and incubation at room temperature for approximately 1 hour. The pH was confirmed after overnight incubation at 4° C., and the pH-adjusted corn stover was autoclaved for 20 minutes at approximately 120° C., and then stored at 4° C. to minimize the risk of microbial contamination. The dry weight of the pretreated corn stover was 33% TS (total solids), which was confirmed before each use using an IR120 moisture analyzer (Denver Instruments, Bohemia, N.Y., USA).

### Example 2

### Pretreatment of Eucalyptus

Unbleached eucalyptus pulp was prepared using a conventional Kraft pulping method. The Kraft pulping liquor contained 17% active alkali (NaOH and Na₂S) with 25% sulfidity. The eucalyptus was cooked by gradually increasing the temperature from room temperature to 160° C. in a digester over 90 minutes. Then the temperature was maintained at 160° C. for another 90 minutes. The eucalyptus pulp was washed extensively with water, and the dry weight determined using an IR120 moisture analyzer.

### Example 3

### Pretreatment of Partially Bleached Eucalyptus

Unbleached eucalyptus pulp was prepared as described in Example 2. The pulp was then further delignified in the following manner. The unbleached pulp was incubated in 1.2% NaOH with 10 bar O₂ for 60 minutes. The partially bleached eucalyptus pulp was then washed extensively with water, and the dry weight determined using an IR120 moisture analyzer.

### Example 4

### Milling Substrates

Lignocellulosic biomass substrates, when not used as unmilled slurries, were milled using a 0.5 HP Attritor Model 01HD with ZrO₂ agitator shaft and arms, and 1400 cc water cooled, jacketed grinding tank (Union Process, Akron, Ohio, USA) with a sealed tank lid. Milling was performed either undiluted, or at 50% (w/w) dilution in deionized water, followed by vacuum filtration using a STERICUP® Filter Unit (Millipore, Billerica, Mass., USA) for at least 6 hours.

Milling was performed in the following manner: approximately 6 pounds of 5 mm ytrrium-stabilized zirconium oxide grinding media were used to charge the grinding tank. An Ecoline RE106 circulating water bath (Lauda, Lauda DR. R. Wobser GMBH & Co., Germany) was used to maintain the tank temperature at 15° C. Approximately 150-200 cc of pH-adjusted, autoclaved pretreated corn stover, autoclaved eucalyptus, or autoclaved bleached eucalyptus were added slowly to the tank with the agitator arms rotating at approximately 200 rpm. The tank was then sealed and the agitator arm velocity increased to approximately 600 rpm. The grinding was permitted to proceed for 15 minutes for pretreated corn stover or for 60 minutes for eucalyptus. Milled biomass substrates were removed from the grinding tank, separated from the grinding media by screening through ¼ inch hardware cloth, and autoclaved.

### Example 5

### Multi-Well Fill Plates

A 96-well plate was generated by machining an aluminum plate of depth ¼ inch with 96, cone-shaped wells, diameter ¼ inch at the upper surface and diameter ⅛ inch at the lower surface. The center of each well was at an equivalent position to the center of a corresponding well in a standard 96-well microtiter plate, approximately 23/64 inch on center. The resulting volume of each well was approximately 135 μl. This 96-well aluminum plate is hereinafter referred to as a “fill plate” or “multi-well fill plate”. A schematic diagram of the multi-well fill plate is shown in FIG. 1.

### Example 6

### Procedure for Transferring High Dry Weight Percentages Lignocellulosic Material to Microtiter Plates and Saccharifying

NREL pretreated corn stover was prepared as described in Example 1. The autoclaved, pH-adjusted corn stover was used to fill the holes in the multi-well fill plate by applying a suitable volume of the corn stover to the upper surface of the plate, then using a spatula to spread the material over the surface and into the holes. Holes were deemed sufficiently full when corn stover was extruded through the hole in the bottom surface, forming noodle-like tubes. A 0.009RD razor blade (American Safety Razor, 1 Razor Blade Lane, Verona, Va., USA) held perpendicular to the fill plate surface was used to scrape excess corn stover from the top and bottom surfaces of the fill plate, leaving the surfaces of the corn stover in each well flush with the surfaces of the fill plate. A Kimwipe (Kimberly Clarke, Roswell, Ga., USA) was used to wipe the excess corn stover from the edges and sides of the fill plate. A 2 ml, 96-deep well plate (Axygen, Union City, Calif., USA) was weighed, and the fill plate was then placed on the top of the deep well plate with the top surface (FIG. 1) adjacent to the open end of the well plate (e.g. the top of the well plate), and the wells aligned with the corn stover-filled holes in the fill plate. The fill plate was secured in this position, and the assembly centrifuged at 2500 rpm (1350×g) for 5 minutes in a Sorvall Legend RT+ (Thermo Scientific, Waltham, Mass., USA). Following centrifugation, the corn stover had been transferred to the deep well plate. Any of the fill-plate holes that still contained corn stover were emptied by pushing a ⅛ inch diameter glass rod through the hole in the back surface of the fill plate to displace the remaining stover into the deep well plate. The deep well plate containing corn stover was reweighed, and the mass of corn stover in the plate determined. The mass of corn stover in each well was determined by dividing the total mass of corn stover by 96. A 3 mm glass bead (Fisher Scientific, Waltham, Mass., USA) was placed in each well for mixing. To rapidly place a single bead in each well, a sheet of plastic with 96 holes typically used to separate pre-racked pipet tips was modified in the following manner. A small groove was ground into the edge of each hole extending away from the hole, each groove in the same direction such that all grooves on the plastic sheet were parallel. To the opposite side of the plastic sheet as the groove, an adhesive sheet was attached. The 3 mm glass beads were poured over the sheet and adhered to the adhesive loosely in each hole. Holes containing more than one bead had excess beads removed. The sheet was inverted on top of the reaction plate, and sliding the sheet displaced the beads, one into each well.

For milled substrates including bleached and unbleached eucalyptus, a heavier gauge spatula or gloved fingertip was used to push the lignocellulosic biomass into fill-plate wells. For fibrous biomass substrates the angle between the razor blade and the fill plate surface was decreased, thereby cutting rather than scraping excess biomass from the fill plate.

The desired final solids content of corn stover was then generated by addition of the appropriate mass of buffer+water+cellulase composition to give the desired dilution factor (e.g., to obtain 20% total solids from a 14.1 g mass of 32.4% total solids NREL pretreated corn stover requires a final mass in each well of 0.238 g, or 0.091 g addition). 1 M Sodium acetate pH 5.0 was added at a suitable volume to generate 50 mM final concentration. The cellulase composition was added to give the final concentration desired. A suitable volume of deionized water was then added to generate the 0.091 g addition required. The buffer, water and cellulase composition were added using multichannel pipets (Rainin Instrument LLC, Oakland, Calif., USA). Plates were sealed using an ALPS 300® plate sealer (ThermoFisher Scientific, Waltham, Mass., USA). A Costar 3099 universal microtiter plate lid (Corning, Corning, N.Y., USA) was placed over the plate seal and affixed with tape. Sealed plates were mixed thoroughly by vigorous shaking, or by inverting the plates and centrifuging upside-down, inverting the plates and centrifuging right-side up and repeating several times as necessary. Finally, each plate was placed into a 150 ml flask adaptor with the springs removed, in an Innova 44 shaker/incubator (New Brunswick Scientific, Edison, N.J., USA) equilibrated at 50° C. and was oriented perpendicular to the plane of the shaker base. This orientation permits greater agitation by the glass beads in each well. Saccharification reactions were incubated with shaking at 150 rpm for 7 days. After 12 to 24 hours, liquification had generally occurred, and plates were removed, shaken vigorously to ensure complete mixing, and returned to the incubator. After 7 days of incubation, the plates were removed from the shaker incubator, cooled to room temperature, centrifuged at 3000 rpm (1940×g), and the seals were removed. High performance liquid chromatography (HPLC) mobile phase buffer, 5 mM H₂SO₄+0.5% (w/w) benzoic acid, was added to each well in a volume necessary to dilute each saccharification reaction 5-fold. Each well was mixed by pipetting, and the supernatants obtained by filtration using a 0.45 μm MULTISCREEN® 96 well centrifuge filter plate (Millipore, Bedford, Mass., USA). Filtered supernatants were analyzed by HPLC.

For HPLC analysis, the sugar concentrations of samples were measured using a 4.6×250 mm AMINEX® HPX-87H column (Bio-Rad Laboratories, Inc., Hercules, Calif., USA) by elution in the HPLC buffer described at a flow rate of 0.6 ml per minute at 65° C. for 11 minutes and quantification by integration of glucose and cellobiose signals from refractive index detection (CHEMSTATION®, AGILENT® 1100 HPLC, Agilent Technologies, Santa Clara, Calif., USA) calibrated with pure sugar samples. The resulting sugar concentrations were used to calculate the fraction or percentage of cellulose conversion for each reaction. The extent of each hydrolysis was determined as the fraction of total cellulose converted to cellobiose+glucose, and was not corrected for soluble sugars present in pretreated corn stover liquor, or was corrected for soluble sugars present in liquor as indicated.

### Example 7

### Correlation to Other High Solids Methods

Fifteen different synthetic mixtures of purified cellulases, hemicellulases, GH61 polypeptides, and beta-glucosidases were generated, varying the composition of the component enzymes between each mixture as shown below in Table 1.

| Mix | CBHI | CBHII | GH61 | Xylanase | BG | BX | Cellulase |
|---|---|---|---|---|---|---|---|
| 1 | 25.00% | 20.00% | 8.00% | 6.00% | 5.00% | 2.00% | 35.00% |
| 2 | 35.00% | 17.30% | 6.50% | 5.20% | 4.30% | 1.30% | 30.30% |
| 3 | 21.90% | 30.00% | 6.60% | 5.30% | 4.40% | 1.30% | 30.60% |
| 4 | 23.00% | 18.40% | 15.00% | 5.50% | 4.60% | 1.40% | 32.20% |
| 5 | 23.90% | 19.10% | 7.20% | 10.00% | 4.80% | 1.40% | 33.50% |
| 6 | 23.70% | 18.90% | 7.10% | 5.70% | 10.00% | 1.40% | 33.20% |
| 7 | 24.60% | 19.70% | 7.40% | 5.90% | 4.90% | 3.00% | 34.50% |
| 8 | 15.40% | 12.30% | 4.60% | 3.70% | 3.10% | 0.90% | 60.00% |
| 9 | 15.00% | 22.70% | 8.50% | 6.80% | 5.70% | 1.70% | 39.70% |
| 10 | 28.10% | 10.00% | 8.40% | 6.80% | 5.60% | 1.70% | 39.40% |
| 11 | 27.00% | 21.60% | 0.00% | 6.50% | 5.40% | 1.60% | 37.80% |
| 12 | 26.10% | 20.90% | 7.80% | 2.00% | 5.20% | 1.60% | 36.50% |
| 13 | 26.30% | 21.10% | 7.90% | 6.30% | 0.00% | 1.60% | 36.80% |
| 14 | 25.40% | 20.30% | 7.60% | 6.10% | 5.10% | 0.00% | 35.50% |
| 15 | 34.60% | 27.70% | 10.40% | 8.30% | 6.90% | 2.10% | 10.00% |

These enzyme mixtures were used to saccharify 20% Total Solids unmilled NREL pretreated corn stover in parallel assays; 20 g scale and the 96 well plate scale as described in Example 5, at two temperatures, 50° C. and 60° C. The concentration of the cellulase composition in the 20 g assay was 5 mg per g cellulose; enzyme concentration in the 96-well plate assay was 4 mg per g cellulose. At 7-days of saccharification, both assays were sampled and HPLC analysis was performed on the filtered hydrolyzates. Correlation coefficients between glucose concentrations obtained in the two assays were determined with the restricted maximum likelihood method using JMP Software (SAS, Cary, N.C., USA).

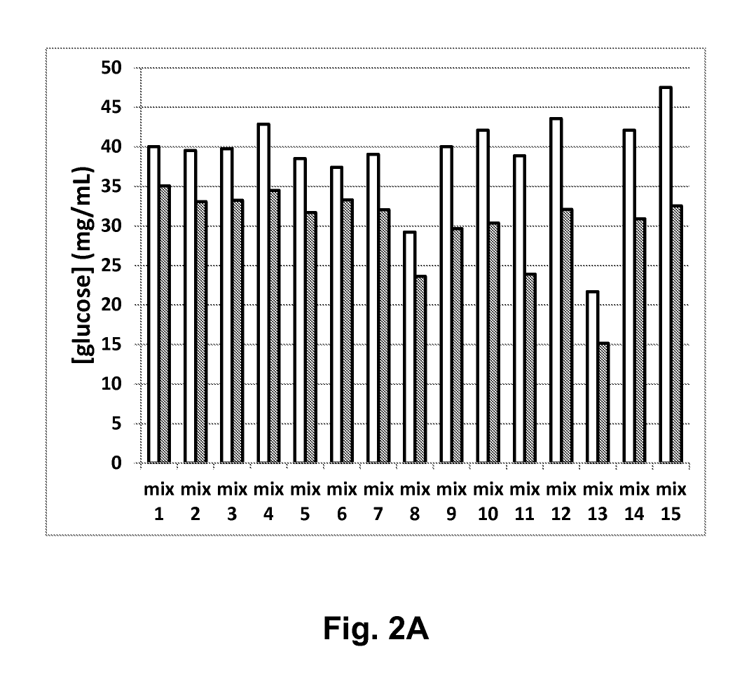

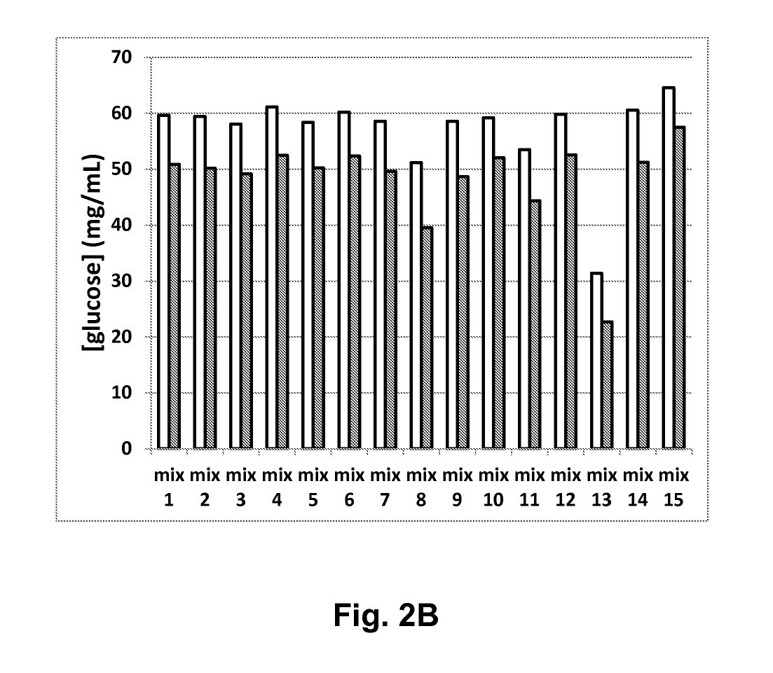

As shown in FIG. 2, substantial variation in glucose yields was observed between cellulase blends in both 96 well and 20 g scale assays, particularly at 50° C. Comparing the relative cellulose conversion by the various mixtures in the 96 well high solids plate (FIG. 2A) to the 20 g scale assays (FIG. 2B), showed clear similarity. The correlation coefficients for glucose concentrations generated by the various cellulase mixtures at both 50° C. and 60° C. in the two different assays were 0.92, indicating a high correlation.

### Example 8

### Various Solids Loading

Saccharification of various dry weight contents of unmilled NREL pretreated corn stover by an enzyme composition composed of a blend of an Aspergillus aculeatus GH10 xylanase (WO 94/021785) and a Trichoderma reesei cellulase preparation containing Aspergillus fumigatus beta-glucosidase (WO 2005/047499) and Thermoascus aurantiacus GH61A polypeptide (WO 2005/074656) (hereinafter “cellulase enzyme composition #1”) was assessed using the 96 well plate assay. The cellulase enzyme composition #1 was dosed at concentrations from 0 to 150 mg per g cellulose by addition of identical volumes of protein to 5 high solids mini-assay plates as described. The solids content in a plate was then varied by diluting the biomass/enzyme slurry to the final concentrations indicated by addition of a suitable volume of buffer and water (Example 6). After 7 days of saccharification, samples were diluted 2.5-fold and analyzed by HPLC as described in Example 6. Data were fit using a modified sigmoidal hydrolysis model.

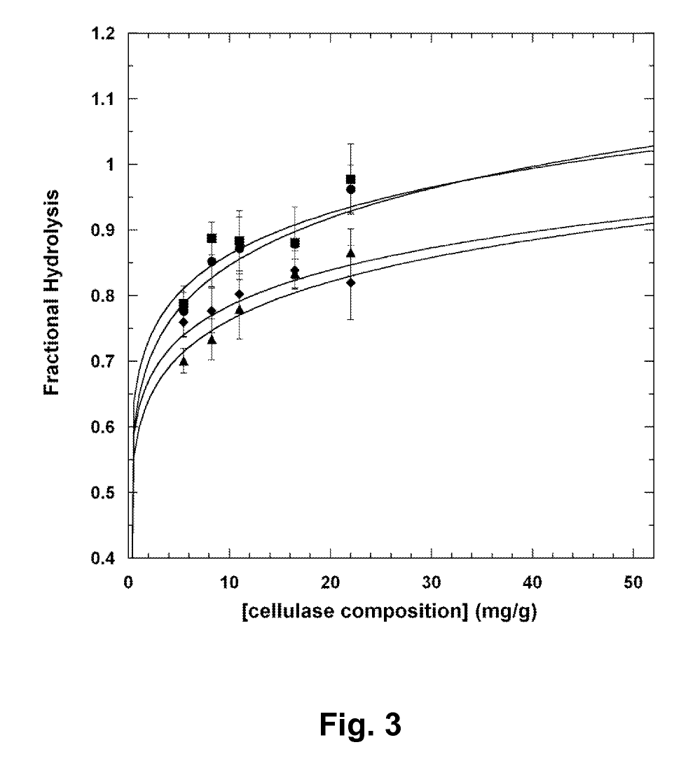

As shown in FIG. 3, as the dry weight percentage was increased, the cellulose conversion that was achieved by a given concentration of cellulase decreased, which was particularly apparent at solids loadings greater than 12.5% TS.

### Example 9

### Comparison of Various Substrates and Cellulase Compositions

Different lignocellulosic substrates were hydrolyzed by a composition composed of a Trichoderma reesei cellulase preparation containing Aspergillus oryzae beta-glucosidase fusion protein (WO 2008/057637) and Thermoascus aurantiacus GH61A polypeptide (WO 2005/074656) (hereinafter cellulase enzyme composition #2) and another composition composed of a blend of an Aspergillus fumigatus GH10 xylanase (WO 2006/078256) and Aspergillus fumigatus beta-xylosidase (WO 2011/057140) with a Trichoderma reesei cellulase preparation containing Aspergillus fumigatus cellobiohydrolase I (WO 2011/057140), Aspergillus fumigatus cellobiohydrolase II (WO 2011/057140), Aspergillus fumigatus beta-glucosidase variant (WO 2012/044915), and Penicillium sp. (emersonii) GH61 polypeptide (WO 2011/041397) (hereinafter “cellulase enzyme composition #3”) using 96-well high solids reactions. The solids content of the reactions was 20% for NREL pretreated corn stover (PCS) or 10% for bleached or unbleached eucalyptus.

Four replicates of each of the 2 cellulase blends at enzyme doses of 0, 2, 4, 6, 8, 10, 12, 14, 16, 18, 20, 22 and 50 mg per g solid were performed for each biomass substrate. Following 7-day saccharifications at 50° C., samples were diluted 5-fold and HPLC analysis was performed as described (Example 6). Data were fit using a modified sigmoidal hydrolysis model using Kaleidagraph (Synergy Software, Reading, Pa., USA).

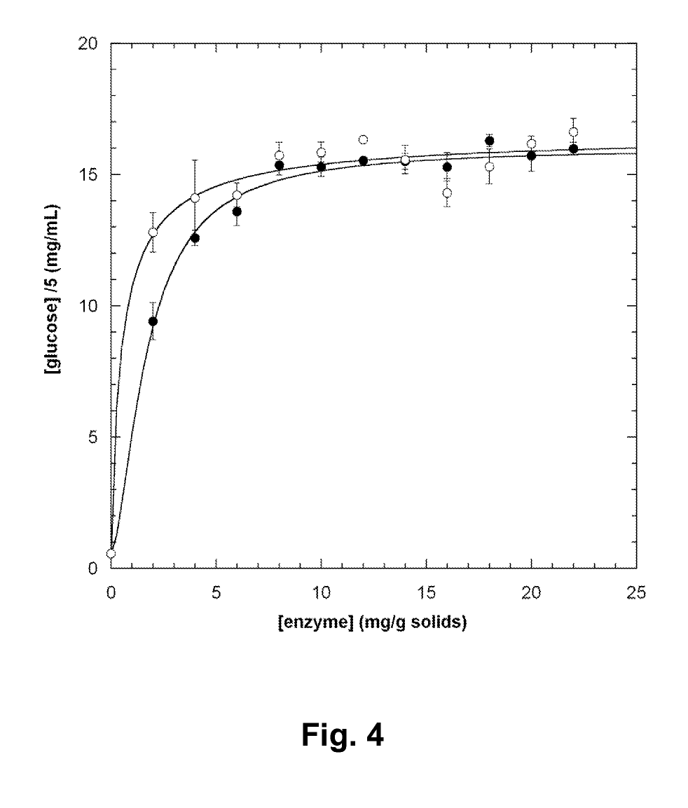

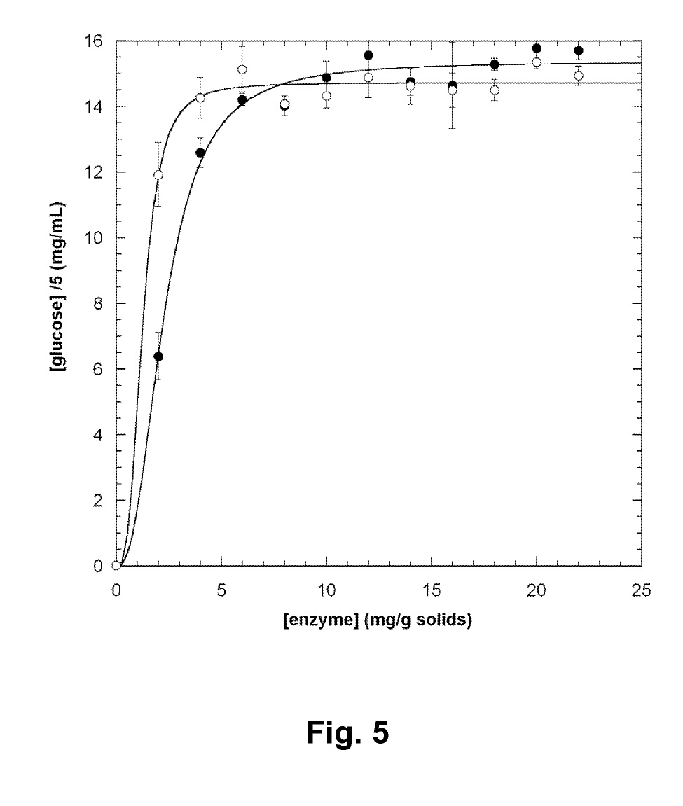

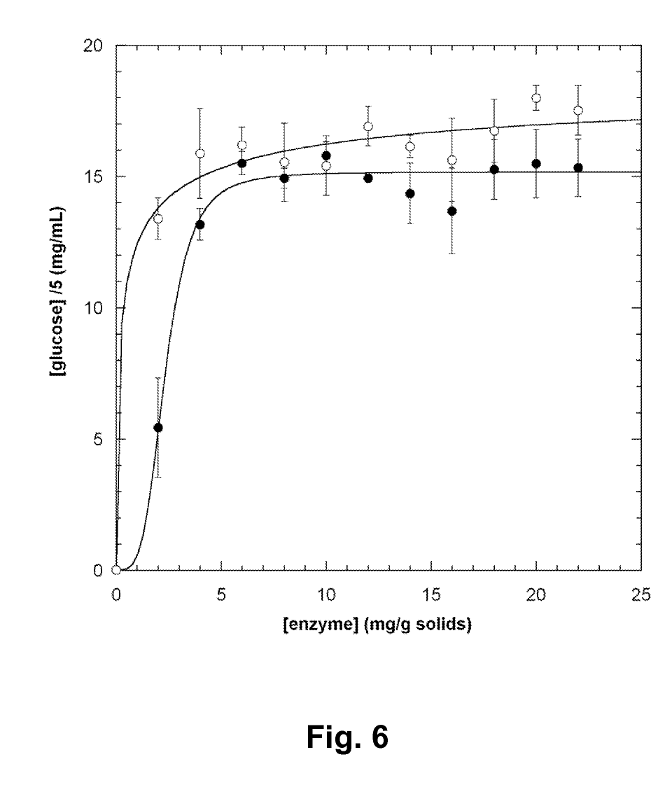

As shown in FIG. 4 (unmilled NREL pretreated corn stover), FIG. 5 (10% for milled bleached eucalyptus), and FIG. 6 (10% for milled unbleached eucalyptus), at lower concentrations of enzyme, cellulase enzyme composition #3 achieved a higher extent of hydrolysis at an equivalent enzyme concentration to cellulase enzyme composition #2. In the case of both eucalyptus substrates 22 mg per gram solids cellulase enzyme composition #3 achieved a greater hydrolysis than did 50 mg per g solids cellulase enzyme composition #2. At the highest concentrations of enzyme, the extent of hydrolysis for both cellulase enzyme compositions was similar for a given substrate.

The present invention is further described by the following numbered paragraphs:

[1] A method for analyzing degradation of a biomass material, comprising: (a) spreading a slurry of the biomass material over a multi-well fill plate to fill each well thereof, wherein each well of the multi-well fill plate comprises an aperture corresponding to a paired opening in the same well; (b) removing excess biomass material from the surfaces of the multi-well fill plate; (c) transferring the biomass material from the wells of the multi-well fill plate to corresponding wells of a multi-well reaction plate by a means that displaces the biomass material from the opening of each well of the multi-well fill plate into the corresponding opening of each well of the multi-well reaction plate, wherein the multi-well reaction plate comprises wells that are larger in volume and larger or equivalent in diameter or width compared to the multi-well fill plate; (d) adding an enzyme composition to each well of the multi-well reaction plate; (e) incubating the reaction plate for a period of time at a pH and temperature for the enzyme composition to degrade the biomass material; and (f) detecting a signal resulting from the degradation of the biomass material, wherein an increase or a decrease in intensity of the signal indicates the amount of biomass material degraded by the enzyme composition.

[2] The method of paragraph 1, wherein the spreading of the slurry of the biomass material over the multi-well fill plate is performed manually, robotically, or mechanically.

[3] The method of paragraph 1 or 2, wherein the volume of each well of the multi-well fill plate is at least 1 μl, e.g., at least 5 μl, at least 10 μl, at least 20 μl, at least 30 μl, at least 40 μl, at least 50 μl, at least 60 μl, at least 70 μl, at least 80 μl, at least 90 μl, at least 100 μl, at least 125 μl, at least 150 μl, at least 175 μl, at least 200 μl, at least 250 μl, at least 500 μl, at least 750 μl, at least 1 ml, at least 2 ml, at least 5 ml, at least 10 ml, at least 25 ml, or at least 50 ml.

[4] The method of any of paragraphs 1-3, wherein the volume of each well of the multi-well reaction plate is at least 1 μl, e.g., at least 5 μl, at least 10 μl, at least 20 μl, at least 30 μl, at least 40 μl, at least 50 μl, at least 60 μl, at least 70 μl, at least 80 μl, at least 90 μl, at least 100 μl, at least 125 μl, at least 150 μl, at least 175 μl, at least 200 μl, at least 250 μl, at least 500 μl, at least 750 μl, at least 1 ml, at least 2 ml, at least 5 ml, at least 10 ml, at least 25 ml, or at least 50 ml.

[5] The method of any of paragraphs 1-4, wherein the volume and diameter or width of each well of the multi-well reaction plate is at least 5%, e.g., at least 10%, at least 20%, at least 30%, at least 40%, at least 50%, at least 100%, at least 500%, at least 1000%, or at least 5000% larger than the volume and diameter or width of each well of the multi-well fill plate.

[6] The method of any of paragraphs 1-5, wherein the removing of the excess biomass material from the surfaces of the multi-well fill plate is performed manually, robotically, or mechanically.

[7] The method of any of paragraphs 1-6, wherein the aperture corresponding to a paired opening in the multi-well fill plate is the same size as the opening of each well.

[8] The method of any of paragraphs 1-6, wherein the aperture corresponding to a paired opening in the multi-well fill plate is smaller in size than the opening of each well.

[9] The method of any of paragraphs 1-6, wherein the aperture corresponding to a paired opening in the multi-well fill plate is a pin hole.

[10] The method of any of paragraphs 1-9, wherein the means that displaces the biomass material from the multi-well fill plate into the multi-well reaction plate is centrifugation.

[11] The method of any of paragraphs 1-9, wherein the means that displaces the biomass material from the multi-well fill plate into the multi-well reaction plate is pressurized gas or negative pressure (i.e., vacuum).

[12] The method of any of paragraphs 1-9, wherein the means that displaces the biomass material from the multi-well fill plate into the multi-well reaction plate is physical displacement.

[13] The methods of any of paragraphs 1-12, wherein the wells of the multi-well fill plate are conical, cylindrical, cubical, oval, rectangular, or spherical in shape.

[14] The methods of any of paragraphs 1-13, wherein the wells of the multi-well reaction plate are conical, cylindrical, cubical, oval, rectangular, or spherical in shape.

[15] The method of any of paragraphs 1-14, wherein the enzyme composition comprises one or more enzymes selected from the group consisting of an amylase, a cellulase, a chitinolytic enzyme, a GH61 polypeptide having cellulolytic enhancing activity, a glucoamylase, a hemicellulase, an esterase, an expansin, a laccase, a ligninolytic enzyme, a pectinase, a peroxidase, a protease, a pullulanase, and a swollenin.

[16] The method of paragraph 15, wherein the cellulase is one or more enzymes selected from the group consisting of an endoglucanase, a cellobiohydrolase, and a beta-glucosidase.

[17] The method of paragraph 15, wherein the hemicellulase is one or more enzymes selected from the group consisting of an acetylmannan esterase, an acetylxylan esterase, an arabinanase, an arabinofuranosidase, a coumaric acid esterase, a feruloyl esterase, a galactosidase, a glucuronidase, a glucuronoyl esterase, a mannanase, a mannosidase, a pectin lyase, a xylanase, and a xylosidase.

[18] The method of any of paragraphs 1-17, wherein the signal detected is selected from the group consisting of reducing sugars (e.g., glucose and/or xylose), disappearance of biomass material, fluidity of the biomass material, viscosity, and absorbance.

[19] The method of any of paragraphs 1-18, further comprising adding a means of mixing to each well of the multi-well reaction plate or to the multi-well reaction plate.

[20] The method of any of paragraphs 1-19, wherein the slurry is at least 1% total solids, e.g., at least 5% total solids, at least 10% total solids, at least 15% total solids, at least 20% total solids, at least 25% total solids, at least 30% total solids, at least 35% total solids, at least 40% total solids, at least 45% total solids, at least 50% total solids, at least 55% total solids, at least 60% total solids, at least 65% total solids, at least 70% total solids, at least 75% total solids, at least 80% total solids, at least 85% total solids, at least 90% total solids, or at least 95% total solids.

[21] The method of any of paragraphs 1-20, wherein the slurry of the biomass material contains one or more internal standards for normalizing differences of the biomass material between the wells.

[22] The method of any of paragraphs 1-21, wherein the biomass material is a cellulosic material, a hemicellulosic material, a starch material, or a chitinous material.

[23] The method of any of paragraphs 1-22, wherein the biomass material is pretreated before use.

[24] The method of any of paragraphs 1-23, further comprising fermenting the hydrolysed or degraded biomass material with one or more (e.g., several) fermenting microorganisms to produce a fermentation product.

[25] A multi-well fill plate for transferring a slurry of a biomass material to a multi-well reaction plate, comprising multiple wells and an aperture corresponding to a paired opening in the multi-well fill plate.

[26] The multi-well fill plate of paragraph 25, wherein the volume of each well of the multi-well fill plate is at least 1 μl, e.g., at least 5 μl, at least 10 μl, at least 20 μl, at least 30 μl, at least 40 μl, at least 50 μl, at least 60 μl, at least 70 μl, at least 80 μl, at least 90 μl, at least 100 μl, at least 125 μl, at least 150 μl, at least 175 μl, at least 200 μl, at least 250 μl, at least 500 μl, at least 750 μl, at least 1 ml, at least 2 ml, at least 5 ml, at least 10 ml, at least 25 ml, or at least 50 ml.

[27] The multi-well fill plate of paragraph 25 or 26, wherein the aperture corresponding to a paired opening in the multi-well fill plate is the same size as the opening of each well.

[28] The multi-well fill plate of paragraph 25 or 26, wherein the aperture corresponding to a paired opening in the multi-well fill plate is smaller in size than the opening of each well.

[29] The multi-well fill plate of paragraph 25 or 26, wherein the aperture corresponding to a paired opening in the multi-well fill plate is a pin hole.

[30] The multi-well fill plate of any of paragraphs 25-29, wherein the wells of the multi-well fill plate are conical, cylindrical, cubical, oval, rectangular, or spherical in shape.

[31] The multi-well fill plate of any of paragraphs 25-30, wherein the multi-well fill plate comprises wells that are smaller in volume and larger or equivalent in diameter or width compared to the multi-well reaction plate for transferring a biomass material from the wells of the multi-well fill plate to corresponding wells of the multi-well reaction plate.

[32] The multi-well plate of any of paragraphs 25-31, wherein the slurry is at least 1% total solids, e.g., at least 5% total solids, at least 10% total solids, at least 15% total solids, at least 20% total solids, at least 25% total solids, at least 30% total solids, at least 35% total solids, at least 40% total solids, at least 45% total solids, at least 50% total solids, at least 55% total solids, at least 60% total solids, at least 65% total solids, at least 70% total solids, at least 75% total solids, at least 80% total solids, at least 85% total solids, at least 90% total solids, or at least 95% total solids.

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

1. A method for analyzing degradation of a biomass material, comprising:
(a) spreading a slurry of the biomass material over a multi-well fill plate to fill each well thereof, wherein each well of the multi-well fill plate comprises an aperture corresponding to a paired opening in the same well;
(b) removing excess biomass material from the surfaces of the multi-well fill plate;
(c) transferring the biomass material from the wells of the multi-well fill plate to corresponding wells of a multi-well reaction plate by a means that displaces the biomass material from the opening of each well of the multi-well fill plate into the corresponding opening of each well of the multi-well reaction plate, wherein the multi-well reaction plate comprises wells that are larger in volume and larger or equivalent in diameter or width compared to the multi-well fill plate;
(d) adding an enzyme composition to each well of the multi-well reaction plate;
(e) incubating the reaction plate for a period of time at a pH and temperature for the enzyme composition to degrade the biomass material; and
(f) detecting a signal resulting from the degradation of the biomass material, wherein an increase or a decrease in intensity of the signal indicates the amount of biomass material degraded by the enzyme composition.

2. The method of claim 1, wherein the volume of each well of the multi-well fill plate is at least 1 μl, e.g., at least 5 μl, at least 10 μl, at least 20 μl, at least 30 μl, at least 40 μl, at least 50 μl, at least 60 μl, at least 70 μl, at least 80 μl, at least 90 μl, at least 100 μl, at least 125 μl, at least 150 μl, at least 175 μl, at least 200 μl, at least 250 μl, at least 500 μl, at least 750 μl, at least 1 ml, at least 2 ml, at least 5 ml, at least 10 ml, at least 25 ml, or at least 50 ml.

3. The method of claim 1, wherein the volume of each well of the multi-well reaction plate is at least 1 μl, e.g., at least 5 μl, at least 10 μl, at least 20 μl, at least 30 μl, at least 40 μl, at least 50 μl, at least 60 μl, at least 70 μl, at least 80 μl, at least 90 μl, at least 100 μl, at least 125 μl, at least 150 μl, at least 175 μl, at least 200 μl, at least 250 μl, at least 500 μl, at least 750 μl, at least 1 ml, at least 2 ml, at least 5 ml, at least 10 ml, at least 25 ml, or at least 50 ml.

4. The method of claim 1, wherein the volume and diameter or width of each well of the multi-well reaction plate is at least 5%, e.g., at least 10%, at least 20%, at least 30%, at least 40%, at least 50%, at least 100%, at least 500%, at least 1000%, or at least 5000% larger than the volume and diameter or width of each well of the multi-well fill plate.

5. The method of claim 1, wherein the aperture corresponding to a paired opening in the multi-well fill plate is the same size as or smaller in size than the opening of each well.

6. The methods of claim 1, wherein the wells of the multi-well fill plate are conical, cylindrical, cubical, oval, rectangular, or spherical in shape.

7. The methods of claim 1, wherein the wells of the multi-well reaction plate are conical, cylindrical, cubical, oval, rectangular, or spherical in shape.

8. The method of claim 1, wherein the signal detected is selected from the group consisting of reducing sugars, disappearance of biomass material, fluidity of the biomass material, viscosity, and absorbance.

9. The method of claim 1, further comprising adding a means of mixing to each well of the multi-well reaction plate or to the multi-well reaction plate.

10. The method of claim 1, wherein the slurry is at least 1% total solids, e.g., at least 5% total solids, at least 10% total solids, at least 15% total solids, at least 20% total solids, at least 25% total solids, at least 30% total solids, at least 35% total solids, at least 40% total solids, at least 45% total solids, at least 50% total solids, at least 55% total solids, at least 60% total solids, at least 65% total solids, at least 70% total solids, at least 75% total solids, at least 80% total solids, at least 85% total solids, at least 90% total solids, or at least 95% total solids.

11. The method of claim 1, wherein the slurry of the biomass material contains one or more internal standards for normalizing differences of the biomass material between the wells.

12. The method of claim 1, wherein the biomass material is pretreated before use.

13. The method of claim 1, further comprising fermenting the hydrolysed or degraded biomass material with one or more fermenting microorganisms to produce a fermentation product.

14. A multi-well fill plate for transferring a slurry of a biomass material to a multi-well reaction plate, comprising multiple wells and an aperture corresponding to a paired opening in the multi-well fill plate.

15. The multi-well fill plate of claim 14, wherein the volume of each well of the multi-well fill plate is at least 1 μl, e.g., at least 5 μl, at least 10 μl, at least 20 μl, at least 30 μl, at least 40 μl, at least 50 μl, at least 60 μl, at least 70 μl, at least 80 μl, at least 90 μl, at least 100 μl, at least 125 μl, at least 150 μl, at least 175 μl, at least 200 μl, at least 250 μl, at least 500 μl, at least 750 μl, at least 1 ml, at least 2 ml, at least 5 ml, at least 10 ml, at least 25 ml, or at least 50 ml.

16. The multi-well fill plate of claim 14, wherein the aperture corresponding to a paired opening in the multi-well fill plate is the same size or smaller in size than as the opening of each well.

17. The multi-well fill plate of claim 14, wherein the wells of the multi-well fill plate are conical, cylindrical, cubical, oval, rectangular, or spherical in shape.
